(19) United States
(12) Patent Application Publication
(10) Pub. No.: US 2026/0028578 A1
(43) Pub. Date: Jan. 29, 2026

(54) DEVICES FOR CELL LYSIS AND METHODS OF USE THEREOF

(71) Applicant: Genzyme Corporation, Cambridge, MA (US)

(72) Inventors: Ethan Lovdal Butler, Cambridge, MA (US); Mario G. Jabra, Cambridge, MA (US); Junfen MA, Cambridge, MA (US); Yuanli Song, Cambridge, MA (US)

(21) Appl. No.: 19/347,877

(22) Filed: Oct. 2, 2025

Related U.S. Application Data
(63) Continuation of application No. PCT/US2024/022746, filed on Apr. 3, 2024.
(60) Provisional application No. 63/456,658, filed on Apr. 3, 2023.
(60) Provisional application No. 63/532,292, filed on Aug. 11, 2023.

(51) Int. Cl. C12M 1/00; C12M 1/12; C12M 3/00

(52) U.S. Cl. CPC C12M 47/06; C12M 23/42; C12M 25/10; C12M 29/04

(57) ABSTRACT

Provided herein are methods for isolating intracellular products (e.g., viral particles or nucleic acid molecules) from a suspension of cells. Also provided herein are also methods for mechanically lysing cells. In some embodiments, provided herein are methods for mechanically lysing cells for isolating intracellular products (e.g., viral particles or nucleic molecules) from a suspension of cells. Also provided herein are apparatuses and cartridges for use in mechanically lysing cells.

### CROSS-REFERENCE TO RELATED PATENT APPLICATIONS

This application claims the priority benefit of U.S. Provisional Application Ser. No. 63/456,658, filed Apr. 3, 2023, and U.S. Provisional Application Ser. No. 63/532,292, filed Aug. 11, 2023, each of which are hereby incorporated by reference in their entirety.

### FIELD

The present invention relates to methods and systems for cell lysis. The present invention also relates to methods and systems for isolating intracellular products such as viral particles or nucleic acid molecules from cells.

### BACKGROUND

Cell lysis is a method used to recover intact intracellular products. There are several cell lysis techniques, such as mechanical or chemical cell lysis, available that can be used across a wide range of cell types. However, not all cell lysis techniques are optimal for lysis efficiency or product recovery. Considerations such as volume and quantity of cells, cost, and the degree of preservation of product are important parameters to consider when selecting a cell lysis technique.

At an industrial scale, large quantities of cells are grown in bioreactors to obtain bulk product. The lysates obtained from cell lysis are subsequently subjected to further downstream processing for product isolation and purification. While cell lysis on small quantities of cells may be easily optimized, the optimal lysis parameters are not scalable.

For example, chemical lysis by surfactant-based methods (e.g., Triton X-100 or Tween-20) is frequently used for large scale product recovery. Surfactants are a class of molecules whose unique properties enable manipulation (disruption or formation) of hydrophobic-hydrophilic interactions among molecules in biological samples. Surfactants are highly efficient as a lysing agent, but lysis techniques require surfactants to be used at a specific weight per volume (w/v) value for optimal lysis. However, surfactant based (e.g., detergent based) cell lysis is variable and inconsistent at various cell densities, thus lysis efficiency does not scale relative to cell number and surfactant concentration. Additionally, while the use of surfactants (e.g., detergent based) may have been required for initial cell lysis or membrane protein extractions, surfactants are difficult to remove from the extracted product, complicating their utility in subsequent applications. Other challenges associated with surfactant based (e.g., detergent based) lysis are increased turbidity, increased filter fouling, extreme reduction in clarification and decreased separation fidelity, higher turbidity, and process/product instability.

To circumvent challenges relating to surfactant-based and/or chemical-based lysis, physical or mechanical disruption techniques may be used for cell lysis. The most common mechanical method used in this field is microfluidization. This method yields more efficient and consistent lysis than chemical methods and is commonly used for nucleic acid production and protein product recovery using E. coli and other ectopic expression systems. However, microfluidization includes extremely high operating pressures (e.g., over 2000 psi). For example, a microfluidizer must be operated at 4000 psi for certain eukaryotic cell types to achieve cell lysis, but this process also damages the product to be recovered. Microfluidizers also require extreme clean-in place protocols between every use to avoid product cross-contamination. In addition, the microfluidizer is challenging to scale, requires high capital expenditures and large investment for various process scales, space and logistical challenges and poses significant logistical challenges when dealing with cells cultivated in large formats such as a bioreactor. Lysis by microfluidization methods may also require harsh chemicals that are not environmentally friendly. These aspects decrease the yield of useful product, which is undesirable especially in the context of biopharmaceuticals.

Thus, there is a need to develop a new mechanical lysing method that does not require high pressures to achieve cell lysis in a sterile manner that is scalable and controllable.

### BRIEF SUMMARY

The inventors have surprisingly discovered that cell lysis and isolation of intracellular products (e.g., polypeptides, viral particles or nucleic acids) from lysed cells can be accomplished using purely mechanical means under gentle conditions that do not compromise the desired isolated product.

In certain aspects, provided herein is a method for isolating viral particles or nucleic acid molecules from a suspension of cells, comprising:
(i) flowing the suspension of cells comprising viral particles or nucleic acid molecules through a cartridge comprising a plurality of fibers or tubes, wherein the fibers or tubes have an inner diameter of no greater than 0.7 mm, and wherein the cells experience a pressure differential of less than 250 psi, thereby lysing a portion of the cells in the suspension to produce a mixed suspension comprising lysed cells and unlysed cells;
(ii) recirculating the mixed suspension formed in step (i) through the cartridge for one or more additional cycles, wherein additional proportions of the cells are lysed at each successive cycle to produce a target suspension comprising lysed cells and cellular debris; and
(iii) separating the viral particles or nucleic acid molecules from the target suspension comprising lysed cells and cellular debris, thereby isolating the viral particles or nucleic acid molecules.

In some embodiments, the suspension of cells comprises producer cells that express the viral particles. In some embodiments, the viral particles are rAAV particles. In some embodiments, the viral particles are produced by a triple transfection method. In some embodiments, the suspension of cells comprises producer cells and the viral particles are produced by triple transfection of the producer cells. In some embodiments, the viral particles are rAAV particles produced by triple transfection method, wherein the triple transfection method comprises transfecting a host cell with one or more plasmids comprising: (i) one or more nucleic acids encoding AAV packaging genes (such as nucleic acid encoding an AAV Rep protein and a nucleic acid encoding an AAV Cap protein); (ii) a nucleic acid encoding a gene of interest; (iii) a nucleic acid encoding at least one Adhelper virus gene. In some embodiments, the viral particles are rAAV particle produced by a transient transfection method. In some embodiments, the viral particles comprise an AAV serotype, wherein the AAV serotype comprises at least one of: AAV1, AAV2, AAV3a, AAV3b, AAV4, AAV5, AAV6, AAV7, AAV8, AAV9, AAV10, AAV11, AAV12, AAV13 and variants thereof, or chimera, or hybrids thereof.

In some embodiments, the viral particles are rAAV particles produced by transfection of the host cell with one or more plasmids comprising: (i) one of more nucleic acids encoding AAV packaging genes (such as nucleic acid encoding an AAV Rep protein and a nucleic acid encoding an AAV Caps protein) and (ii) a nucleic acid encoding a gene of interest. In some embodiments, the viral particles are rAAV particles produced by transfection of the host cell with one or more plasrnids comprising: (i) one of more nucleic acids encoding AAV packaging genes (such as nucleic acid encoding an AAV Rep protein and a nucleic acid encoding an AAV Caps protein); (ii) a nucleic acid encoding a gene of interest; (iii) one or more nucleic acids encoding AAV ITRs (inverted terminal repeats) (e.g., two AAV ITRs), and optionally (iv) a nucleic acid encoding at least one Adhelper virus gene. In some embodiments, the Adhelper virus gene is a helper gene. In some embodiments, the viral particles are rAAV particles expressed from a producer cell line (PCL). In some embodiments, the PCL comprises a host cell stably integrated with: (i) one of more nucleic acids encoding AAV packaging genes (such as nucleic acid encoding an AAV Rep protein and a nucleic acid encoding an AAV Caps protein): (ii) a nucleic acid encoding a gene of interest; (iii) one or more nucleic acids encoding AAV ITRs (inverted terminal repeats) (e.g., two AAV ITRs). In some embodiments, the viral particles are rAAV particles expressed from a PCL that is further infected with an Adhelper virus (e.g., an adenovirus) that expresses one of more helper genes. In some embodiments, the viral particles are rAAV particle produced by a transient transfection method. In some embodiments, the viral particles are lentiviral particles. In some embodiments, the viral particles comprise a recombinant baculovirus expression vector. In some embodiments, the viral particles are rAAV particles expressed from a producer cell line (PCL). In some embodiments, the PCL comprises a host cell stably integrated with: (i) one of more nucleic acids encoding AAV packaging genes; (ii) a nucleic add encoding a gene of interest: (iii) one or more nucleic adds encoding AAV TTRs (inverted terminal repeats). In some embodiments, the PCL is further infected with an adenovirus. In some embodiments, the adenovirus is an Adhelper virus. In some embodiments, the Adhelper virus expresses one of more helper genes. In some embodiments, the AAV packaging genes comprise at least a AAV capsid (cap) gene. In some embodiments, the AAV packaging genes comprise at least a AAV replication (rep) gene.

In some embodiments, the cartridge comprises a plurality of hollow fibers, wherein the fibers comprise a lumen side and a shell side. In some embodiments, the plurality of hollow fibers are configured into a hollow fiber membrane module, wherein the plurality of hollow fibers are grouped together to form a hollow fiber bundle, and wherein the hollow fiber bundle is packed into a tube shell.

In some embodiments, the inner diameter of the fibers comprising the hollow fiber membrane is from 0.1 mm to about 0.7 mm. In some embodiments, the inner diameter of the fibers comprising the hollow fiber membrane is from 0.1 mm to about 5 mm. In some embodiments, the inner diameter of the fibers comprising the hollow fiber membrane is from 0.2 mm to about 0.3 mm.

In some embodiments, the inner diameter of the fibers comprising the hollow fiber membrane is about 0.2 mm. In some embodiments, the inner diameter of the fibers comprising the hollow fiber membrane is about 0.25 mm. In some embodiments, the inner diameter of the fibers comprising the hollow fiber membrane is about 0.3 mm.

In some embodiments, the cells flowing through the cartridge experience a pressure differential of less than 200 psi. In some embodiments, the cells flowing through the cartridge experience a pressure differential of less than 150 psi. In some embodiments, the cells flowing through the cartridge experience a pressure differential of less than 100 psi. In some embodiments, the cells flowing through the cartridge experience a pressure differential of less than 50 psi. In some embodiments, the cells flowing through the cartridge experience a pressure differential of from about 5 psi to about 40 psi. In some embodiments, the cells flowing through the cartridge experience a pressure differential of from about 10 psi to about 40 psi. In some embodiments, the cells flowing through the cartridge experience a pressure differential of from about 15 psi to about 35 psi. In some embodiments, the cells flowing through the cartridge experience a pressure differential of from about 20 psi to about 30 psi. In some embodiments, the cells flowing through the cartridge experience a pressure differential of about 20 psi. In some embodiments, the cells flowing through the cartridge experience a pressure differential of about 22 psi. In some embodiments, the cells flowing through the cartridge experience a pressure differential of about 30 psi. In some embodiments, the cells flowing through the cartridge experience a pressure differential of about 37 psi. In some embodiments, the cells flowing through the cartridge experience a pressure differential of about 50 psi. In some embodiments, the pressure differential is defined by the difference in pressure at the cartridge inlet and pressure at the cartridge outlet.

In some embodiments, the suspension is flowed through the cartridge at a flow rate. In some embodiments, the flow rate is from about 10 L per minute to about 100 L per minute. In some embodiments, the flow rate is from about 50 mL per minute to about 100 L per minute. In some embodiments, the flow rate is 200 mL per minute. In some embodiments, the flow rate is 360 mL per minute. In some embodiments, the flow rate is 600 mL per minute. In some embodiments, the suspension is flowed through the cartridge at a flow rate determined by the cartridge size. In some embodiments, the suspension is flowed through the cartridge at a flow rate determined by the pressure at the cartridge inlet. In some embodiments, the suspension is flowed through the cartridge at a shear rate of at least about 2000 s⁻¹. In some embodiments, the fibers have a thickness of from about 0.1 mm to about 0.4 mm.

In some embodiments, the length of the fibers is from about 20 cm to about 150 cm.

In some embodiments, step (ii) comprises re-circulating the mixed suspension of partially lysed cells formed in step (i) through the cartridge for at least 1 cycle. In some embodiments, step (ii) comprises re-circulating the mixed suspension of partially lysed cells formed in step (i) through the cartridge for at least 5 cycles. In some embodiments, step (ii) comprises re-circulating the mixed suspension of partially lysed cells formed in step (i) through the cartridge for at least 10 cycles. In some embodiments, step (ii) comprises re-circulating the mixed suspension of partially lysed cells formed in step (i) through the cartridge for at least 20 cycles. In some embodiments, step (ii) comprises re-circulating the mixed suspension of partially lysed cells formed in step (i) through the cartridge for at least 30 cycles. In some embodiments, step (ii) comprises re-circulating the mixed suspension of partially lysed cells formed in step (i) through the cartridge for at least 40 cycles. In some embodiments, step (ii) comprises re-circulating the mixed suspension of partially lysed cells formed in step (i) through the cartridge for from about 1 cycle to about 50 cycles. In some embodiments, step (ii) comprises re-circulating the mixed suspension of partially lysed cells formed in step (i) through the cartridge for from about 10 cycles to about 50 cycles. In some embodiments, step (ii) comprises re-circulating the mixed suspension of partially lysed cells formed in step (i) through the cartridge for from about 15 cycles to about 40 cycles. In some embodiments, step (ii) comprises re-circulating the mixed suspension of partially lysed cells formed in step (i) through the cartridge for from about 20 cycles to about 30 cycles.

In some embodiments, the suspension or the mixed suspension of cells is flowed through the lumen side. In some embodiments, the space between the hollow fiber bundle and the tube shell is capped.

In some embodiments, the total time to complete steps (i) and (ii) is from about 1 hour to about 4 hours.

In some embodiments, greater than 70% lysing efficiency is achieved, wherein lysing efficiency is measured by lactate dehydrogenase release, viable cell count percentage, or product release percentage per number of cycles. In some embodiments, greater than 80% lysing efficiency is achieved. In some embodiments, greater than 90% lysing efficiency is achieved.

In some embodiments, the cells are eukaryotic cells. In some embodiments, the cells are mammalian cells. In some embodiments, the cells are derived from a human or a human cell line, and the human cell or human cell line is HEK293 or HeLa. In some embodiments, the mammalian cells are HeLa cells. In some embodiments, the mammalian cels are HEK293 cells. In some embodiments, the mammalian cells are at least one of: CHO cells, HEK293 cells, Vero cells, HeLa cells, MDCK cells, BHK cells, or A549 cells. In some embodiments, the cells are insect cells. In some embodiments, the cells are yeast cells. In some embodiments, the cells are prokaryotic cells. In some embodiments, the prokaryotic cells are E. coli cells.

In some embodiments, lysing is determined by viable cell count percentage. In some embodiments, the viable cell count percentage is from about 20% to about 99% after 5 cycles. In some embodiments, viable cell count percentage is from about 5% to about 90% after 10 cycles. In some embodiments, the viable cell count percentage is from about 1% to about 70% after 20 cycles.

In some embodiments, lysing is determined by viable cell density percentage. In some embodiments, the viable cell density percentage is from about 20% to about 99% after 5 cycles. In some embodiments, the viable cell density percentage is from about 5% to about 90% after 10 cycles. In some embodiments, the viable cell density percentage is from about 1% to about 70% after 20 cycles.

In some embodiments, lysing is determined by percent lysis. In some embodiments, percent lysis is 80% after 18 cycles. In some embodiments, percent lysis is 80% after 11 cycles. In some embodiments, percent lysis is 80% after 8 cycles. In some embodiments, percent lysis is 80% after 5 cycles.

In some embodiments, the suspension of cells is flowed through a container in each successive cycle. In some embodiments, the container is a bioreactor. In some embodiments, the bioreactor is set to a temperature. In some embodiments, the temperature is at about 25° C. to about 37° C. In some embodiments, the bioreactor is at 25° C. In some embodiments, the bioreactor is at 37° C. In some embodiments, the cartridge and the container are operably linked to form a closed system. In some embodiments, the closed system further comprises at least one tubing line that is operably linked to the container and the cartridge, wherein the cell suspension flows between the container and the cartridge through the tubing. In some embodiments, the tubing is rubber.

In some embodiments, separating comprises affinity capture. In some embodiments, separating comprises ion exchange. In some embodiments, separating comprises anion exchange.

In certain aspects, provided herein is a method for mechanically lysing cells, comprising flowing a suspension comprising a plurality or a population of cells through a cartridge comprising a plurality of fibers or tubes, wherein the cells experience a pressure differential of less than 250 psi, thereby lysing a portion of the cells in the suspension.

In some embodiments, the cells are further recirculated through the cartridge for a number of cycles to increase the proportion of cells that are lysed.

In some embodiments, the cells are introduced to the cartridge by following a flow path.

In some embodiments, mechanically lysing comprises shearing the cells. In some embodiments, mechanical cell lysis is achieved by applying one or more external forces to the plurality or population of cells. In some embodiments, the one or more external forces is selected from the group consisting of flow, pressure, turbulence, friction, extrusion, collision, and pressure drop.

In some embodiments, the plurality of fibers are hollow fibers. In some embodiments, the plurality of hollow fibers are configured into a hollow fiber membrane module, wherein the plurality of hollow fibers are grouped together to form a hollow fiber bundle, and wherein the hollow fiber bundle is packed into a tube shell. In some embodiments, the hollow fiber membrane bundle is surrounded by a shell extending longitudinally along the length of the bundle, forming a lumen side and a shell side of the hollow fiber membrane module. In some embodiments, the lumen forms a tubular structure. In some embodiments, the space between the hollow fiber bundle and the tube shell is capped. In some embodiments, the hollow fiber membrane module is sterile. In some embodiments, the hollow fiber membrane module is single-use.

In some embodiments, the number of cycles is determined by the formula

$${N}_{cycle} = \frac{{q}_{feed} t}{{V}_{reactor}}$$

wherein q_{feed} is the flow rate going through the hollow-fiber module, t is operation time and V_{reactor} is volume of aqueous solution comprising the plurality or population of cells.

In some embodiments, the total lysing time is from approximately 1 hour to about 4 hours.

In some embodiments, the portion of cells lysed is greater than 90%.

In certain aspects, provided herein is an apparatus for lysing cells, comprising a container capable of holding a suspension of cells and a cartridge comprising a plurality of fibers or tubes and an extracapillary space outside the fibers or tubes, and a means of connecting the container to the cartridge such that the container and cartridge form a closed system such that when operable, a suspension of cells flows multiple times through the container and the cartridge.

In some embodiments, the means for connecting the container to the cartridge comprises a tubing. In some embodiments, the fibers or tubes have a diameter up to about 0.7 mm.

In some embodiments, the fibers or tubes have a diameter from about 0.1 mm to about 0.5 mm. In some embodiments, the fibers or tubes have a diameter from about 0.2 mm to about 0.3 mm. In some embodiments, the container is a bioreactor.

In certain aspects, provided herein is a method for isolating viral particles or nucleic acid molecules from cells, comprising:
(i) flowing a suspension of cells comprising viral particles or nucleic acid molecules through a cartridge comprising a fibrous porous medium or material, thereby lysing a portion of the cells in the suspension to produce a mixed suspension comprising lysed cells and unlysed cells;
(ii) recirculating the mixed suspension of partially lysed cells formed in step (i) through the cartridge for one or more additional cycles, wherein additional portions of the cells are lysed at each successive cycle to produce a target suspension comprising lysed cells and cellular debris; and
(iii) separating the viral particles or nucleic acid molecules from the target suspension comprising lysed cells and cellular debris, thereby isolating the viral particles or nucleic acid molecules.

In some embodiments, the fibrous porous medium or material is a nonwoven material. In some embodiments, the nonwoven material is a spunbound, melt-blown fiber, felt, or wet-laid material.

In some embodiments, the fibrous porous medium or material is a woven material. In some embodiments, the fibrous porous medium or material is a membrane.

In some embodiments, the nonwoven material enables retention of at least about 90% of lysed cells and cellular debris comprising a size at least about 0.1 μm.

In some embodiments, the pore size of the nonwoven is from about 20 μm to about 60 μm.

In certain aspects, provided herein is a cartridge for lysing cells, comprising
(a) a housing and
(b) a plurality of pores, wherein the pores have a diameter up to or about 1 mm.

In some embodiments, the pores are embedded within a plurality of hollow fibers having an inner diameter, an outer diameter, two ends with a length between said ends, at least one end of each fiber open for fluid entrance or exit, said fibers arranged parallel one another.

In some embodiments, the fibers or tubes do not have pores. In some embodiments, the fibers or tubes comprise pores. In some embodiments, the pores are less than 300 kDa.

In some embodiments, the viral particles are produced by a transient transfection method. In some embodiments, the suspension of cells comprises producer cells that express the viral particles. In some embodiments, the viral particles comprise an AAV serotype, wherein the AAV serotype comprises at least one of: AAV1, AAV2, AAV3a, AVV3b, AAV4, AAV5, AAV6, AAV7, AAV8, AAV9, AAV10, AAV11, AAV12, AAV13 and variants thereof, or chimeras, or hybrids thereof.

In some embodiments, the cells are mammalian cells. In some embodiments, the mammalian cells are at least one of: CHO cells, HEK293 cells, Vero cells, HeLa cells, HEK293 cells, MDCK cells, BHK cells, or A549 cells.

### BRIEF DESCRIPTION OF FIGURES

FIG. 1A is a schematic of a hollow fiber membrane module.

FIG. 1B shows the flow of buffer and a suspension of cells through a hollow fiber membrane module. The semi-circles indicate cell lysis.

FIG. 1C is a schematic showing the flow of buffer and a suspension of cells through a porous membrane. The semi-circles indicate cell lysis.

FIG. 2A is a schematic of a container that is operably linked to a cartridge such that the container and the cartridge forms a closed system.

FIG. 2B is a schematic of a container operably linked to a hollow fiber membrane module.

FIG. 2C is a schematic of a bioreactor operably linked to a hollow fiber membrane module. The Xs indicate capped outlets.

FIG. 2D is a schematic of a bioreactor operably linked to a hollow fiber membrane module. A cross-section of the hollow fiber membrane module is shown. The Xs indicate capped outlets.

FIG. 3 shows the viable cell count of HEK-293 lysed at various cycles following circulating the HEK-293 cells through a hollow fiber (HF) membrane.

FIG. 4 shows percentage lysed according to LDH, viable cell count percentage and product (rAAV) release percentage as a function of cycle number. rAAV release (VG release; square) and LDH release according to microfluidization, as well as VG release using the surfactant PS-20 (triangle) was used as controls.

FIG. 5 shows viable cell count percentage as a function of cycle number using a hollow fiber membrane module at 30 psi using HeLa cells.

FIG. 6 shows viable cell count percentage as a function of cycle number using a nonwoven material with 40 μm at 30 psi using HeLa cells.

FIG. 7 shows percentage lysis as a function of cycle number using hollow fiber membrane modules (Cytiva™ and Planova™ modules) at 20 psi using HEK293 cells.

FIG. 8A shows percentage cells lysed and AAV viral genome (vg) release percentage as a function of cycle number for the 500 L reactor run using a Cytiva™ hollow fiber membrane module for HEK293 cells.

FIG. 8B shows the viable cell density as a function of mechanical cell lysis cycles. Mechanical cell lysis was performed for 500 L for a total of 6 cycles.

FIG. 8C shows percent viability for three different cell lines undergoing mechanical lysis. MCL was performed for 500 L of cells, using a Cytiva™ hollow fiber with a 150 cm² membrane area operated at a pressure of 30 psi and at 15 minutes per cycle.

FIG. 9 shows viable cell count percentage as a function of cycle number for HEK293 cells across various conditions (Trains 1 to 6).

FIG. 10A shows the relationship between the differential pressure and amount of throughput after mechanical cell lysis (MCL) or PS-20 (surfactant) based lysis. MCL was performed for 15 cycles.

FIG. 10B shows the relationship between permeate flux and throughput after mechanical cell lysis (MCL) or PS-20 (surfactant) based lysis. Lysate was subjected to time to filter (TTF) experiments after 3 days after lysis at 4° C. NTU is Nephelometric Turbidity Units. MCL was performed for 15 cycles.

FIG. 11 shows a chromatogram of the recombinant AAV purified from cells lysed by mechanical cell lysis. Protein was quantified using absorbance at 280 nm (UV 280); nucleic acid was quantified using absorbance at 254 (UV 254).

FIG. 12 shows a chromatogram depicting the size of the recombinant AAV purified from cells lysed by mechanical cell lysis.

FIG. 13 shows dynamic light scattering plots of the recombinant AAV purified from cells lysed by mechanical cell lysis or PS-20. Nucleic acid was quantified using absorbance at 260 (UV 260).

FIG. 14A shows percentage lysed and the corresponding relative titer increase of released rAAV particles using mechanical cell lysis methods for transient transfection cells (Platform 1A), compared to the unlysed sample (control). MCL was performed for 5 L of cells, using a Cytiva™ hollow fiber with a 150 cm² membrane area operated at a pressure of 30 psi.

FIG. 14B shows percentage lysed and the corresponding relative titer increase of released rAAV particles using mechanical cell lysis methods for cell Platform 2, compared to the unlysed sample (control). MCL was performed for 5 L of cells, using a Cytiva™ hollow fiber with a 150 cm² membrane area operated at a pressure of 30 psi.

FIG. 15 shows percent viability of cells over time when lysed using MCL at various temperatures. Cycle length was 15 minutes per cycle.

FIG. 16 shows the amount of full capsid recovered after MCL executed at various temperatures, followed by affinity purification and anion exchange. Percent full capsid was determined by mass photometry (MP).

FIG. 17A shows the percent lysed (%) of a HeLa producer cell line platform (Platform 3) cells over multiple cycles of mechanical cell lysis. Mechanical cell lysis was performed using 20 cm² Repligen™ hollow fiber modules (PES, 750 MWCO). The experiments were performed at pressures of 45-50 psi at 37° C.

FIG. 17B shows percent viable cell density (VCD %) of a HeLa producer cell line platform (Platform 3) cells over multiple cycles of mechanical cell lysis. Mechanical cell lysis was performed using 20 cm² Repligen™ hollow fiber modules (PES, 750 MWCO). The experiments were performed at pressures of 45-50 psi at 37° C.

FIG. 18 shows percent lysed (%) of various HeLa cells lines subjected to different pressure drops over multiple cycles of mechanical cell lysis. Mechanical cell lysis was performed using 20 cm² Repligen™ hollow fiber modules (PES, 750 MWCO).

FIG. 19 shows the number of cycles to reach 80% percent lysis of HEK293 cells (Platform 1). Mechanical cell lysis was performed using 20 cm² Repligen™ hollow fiber modules (PES, 750 MWCO).

### DETAILED DESCRIPTION

Provided herein are methods for isolating intracellular product (e.g., viral particles or nucleic acid molecules) from a suspension of cells. Also provided herein are methods for mechanically lysing cells. In some embodiments, provided herein are methods for mechanically lysing cells for isolating viral particles or nucleic molecules from a suspension of cells. Also provided herein are apparatuses and cartridges for use in mechanically lysing cells.

Existing methods for isolating intracellular product (e.g., viral particles or nucleic acid molecules) from a suspension of cells are inconsistent, not scalable, and may unintentionally damage the product during extraction. Therefore, the methods described herein are useful for lysing cells to recover the viral particles or nucleic acid molecules under less stringent conditions (e.g., lower pressure) compared to existing chemical and mechanical lysis techniques.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As utilized in accordance with the present disclosure, the following terms, unless otherwise indicated, shall be understood to have the following meanings. Unless otherwise required by context, singular terms shall include pluralities and plural terms shall include the singular. As used in this specification and the appended claims, the singular forms “a”, “an” and “the” include plural referents unless the content clearly dictates otherwise. Thus, for example, reference to “a molecule” optionally includes a combination of two or more such molecules, and the like.

It is understood that aspects and embodiments of the present disclosure described herein include “comprising,” “consisting,” and “consisting essentially of” aspects and embodiments.

The term “polynucleotide” as used herein refers to single-stranded or double-stranded nucleic acid polymers of at least 10 nucleotides in length. In certain embodiments, the nucleotides comprising the polynucleotide can be ribonucleotides or deoxyribonucleotides or a modified form of either type of nucleotide. The term “polynucleotide” specifically includes single-stranded and double-stranded forms of DNA and/or RNA.

An “isolated” protein or polypeptide is one that has been identified and separated and/or recovered from a component of its natural environment. Contaminant components of its natural environment are materials that would interfere with diagnostic or therapeutic uses for the binding protein, and may include enzymes, hormones, and other proteinaceous or non-proteinaceous solutes.

The terms “substantially pure” or “substantially purified” as used herein refer to a compound or species that is the predominant species present {i.e., on a molar basis it is more abundant than any other individual species in the composition). In some embodiments, the species (e.g., virus or nucleic acid) is purified to essential homogeneity.

By “vector” is meant any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

By an “AAV vector” is meant a vector derived from an adeno-associated virus serotype, including without limitation, AAV-1, AAV-2, AAV-3, AAV-4, AAV-5, AAV-6, AAV-7 and AAV-8. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

The terms “recombinant AAV virion,” “rAAV virion,” “AAV vector particle,” “full capsids,” “fulls,” and “full particles” are defined herein as an infectious, replication-defective virus including an AAV protein shell, encapsidating a heterologous nucleotide sequence of interest which is flanked on both sides by AAV ITRs. In some instances, a rAAV virion is produced in a suitable host cell which has had sequences specifying an AAV vector, AAV helper functions and accessory functions introduced therein. In this manner, the host cell is rendered capable of encoding AAV polypeptides that are required for packaging the AAV vector (containing a recombinant nucleotide sequence of interest) into infectious recombinant virion particles for subsequent gene delivery.

“Operably linked” refers to an arrangement of elements wherein the components so described are functionally connected in a manner that also allows them to perform their usual function.

The term “fiber geometry” or “hollow-fiber geometry” refers to physical properties relating to the fibers, tubes, hollow-fiber filter fibers, or hollow-fiber tubes. Physical properties include, but are not limited to fiber or tube material, length, pore size, pore shape, diameter, surface area, and surface roughness.

The term “full capsid titer” as used in reference to a viral titer, refer to the number of virions containing the recombinant AAV DNA genome, regardless of infectivity or functionality. In some embodiments, the full capsid titer of a viral vector may be measured in terms of vg/mL. Methods suitable for measuring this titer are known in the art (e.g., quantitative PCR).

The term “total capsid titer” as used in reference to a viral titer, refer to the number of virions or AAV protein shells, regardless of infectivity or functionality. In some embodiments, the capsid titer of a viral vector may be measured in terms of cp/mL. Methods suitable for measuring total capsid titer are known in the art (e.g., ELISA).

### Methods of Mechanically Lysing Cells

The methods described herein provide for a method for mechanically lysing cells. In some embodiments, the method comprises flowing a suspension comprising a plurality or a population of cells through a cartridge comprising a plurality of fibers or tubes. In other embodiments, the method comprises flowing a suspension comprising a plurality or a population of cells through a cartridge comprising a fibrous porous material.

The flow path of a system is dictated by the openings of the closed system as disclosed herein. In some embodiments, the cells are introduced to the cartridge by following a flow path.

Mechanical cell lysis (e.g., mechanically lysing cells or mechanical lysis of cells) is breaking cellular membranes by using one or more mechanical forces. A mechanical force is a physical activity that disrupts a cell by breaking cellular membranes. Mechanical forces that may be used according to mechanical lysis may include high shear fluid forces. In some embodiments, mechanical cell lysis is the breaking of cellular membranes by using shear force (e.g., shear stress). In some embodiments, mechanically lysing comprises shearing the cells. In some embodiments, mechanical cell lysis is achieved by applying one or more external forces to the plurality or population of cells.

In some implementations, the cells may be placed under flow within a closed system. The flow applies a shear stress to the cell. Shear stress is the mechanical force induced by the friction of liquid against the apical cell membrane. In some embodiments, the external force is flow. In some implementations, the cells may experience pressure, which is the ratio of force applied over an area (e.g., the area of the cell). In some embodiments, the external force is pressure. Turbulence is the fluid motion characterized by chaotic changes in pressure and flow velocity. In some embodiments, the external force is turbulence. Cell extrusion is the act of squeezing a cell, for example out of a pore or a membrane. In some embodiments, the external force is extrusion. Collisions may be represented by, for example, one or more cells that contact each other at high velocity. In some embodiments, the external force is collision. Pressure drop is the difference in total pressure between two points in a fluid-carrying network. In some embodiments, the external force is pressure drop. In some embodiments, the one or more external forces is selected from the group consisting of flow, pressure, turbulence, friction, extrusion, collision, and pressure drop. The forces may be applied between buffer and the cell, the cell and another cell, the fiber and the cell, the membrane and the cell, or any combination thereof. In some embodiments, the one or more external forces is determined by hollow-fiber geometry.

The methods described herein also provide for a method for mechanically lysing cells under gentle conditions that do not compromise the integrity of desired intracellular products. In some embodiments, the method for mechanically lysing cells is as efficient in extracting intracellular product from cells compared to a method for detergent based cell lysis. In some embodiments, the method for mechanically lysing cells is more efficient in extracting intracellular product compared to a method for detergent based cell lysis. Efficiency may be measured by parameters such as intact product recovery or product purity, by standard biochemical and biophysical assays, such as those disclosed herein. In some embodiments, the surfactant (e.g., detergent) based cell lysis method uses PS-20. In some embodiments, the method comprises flowing a suspension comprising a plurality or a population of cells through a cartridge comprising a plurality of fibers or tubes, wherein the cells experience a pressure differential of less than 1,000 psi, less than 500 psi, less than 250 psi, less than 100 psi, less than 50 psi, or less than 25 psi, thereby lysing a portion of the cells. In some embodiments, the pressure differential is determined by hollow-fiber geometry. In some embodiments, the cells are further recirculated through the cartridge for a number of cycles to increase cell lysis.

In some embodiments, the pressure differential is defined by the difference in pressure at the cartridge inlet and pressure at the cartridge outlet. In some embodiments, the suspension of cells flowing through the cartridge experience a pressure differential of less than 250 psi. In some embodiments, the pressure differential is less than 200 psi. In some embodiments, the pressure differential is less than 150 psi. In some embodiments, the pressure differential is less than 100 psi. In some embodiments, the pressure differential is less than 50 psi. In some embodiments, the pressure differential is from about 5 psi to about 40 psi. In some embodiments, the pressure differential is from about 10 psi to about 40 psi. In some embodiments, the pressure differential is from about 15 psi to about 35 psi. In some embodiments, the pressure differential is from about 20 psi to about 30 psi. In some embodiments, the pressure differential is about 20 psi. In some embodiments, the pressure differential is about 22 psi. In some embodiments, the pressure differential is about 30 psi. In some embodiments, the pressure differential is about 37 psi. In some embodiments, the pressure differential is about 50 psi. In some embodiments, the pressure differential is less than 250 psi, thereby lysing a portion of the cells in the suspension to produce a suspension of partially lysed cells. In some embodiments, the pressure differential is determined by hollow-fiber geometry.

In some embodiments, the cells are further recirculated through the cartridge for a number of cycles to increase cell lysis. In some embodiments, the number of cycles is determined empirically. In some embodiments, the number of cycles is not determined by the total starting number of cells. In some embodiments, the number of cycles is determined by the formula

$${N}_{cycle} = \frac{{q}_{feed} t}{{V}_{reactor}}$$

wherein q_{feed} is the flow rate going through the hollow-fiber module, t is operation time and V_{reactor} is volume of aqueous solution comprising the plurality or population of cells. In some embodiments, the total operation time is from approximately 1 hour to about 4 hours. In some embodiments, the total operation time is the total lysing time. In some embodiments, the total lysing time is from approximately 1 hour to about 4 hours. In some embodiments, the cells are further recirculated through the cartridge for a number of cycles to increase the proportion of cells that are lysed. In some embodiments, the cells are introduced to the cartridge by following a flow path. In some embodiments, the flow path is the flow path disclosed herein. In some embodiments, mechanical lysis comprises shearing the cells. In some embodiments, mechanical cell lysis is achieved by applying one or more external forces to the plurality or population of cells. In some embodiments, the one or more external forces is selected from the group consisting of flow, pressure, turbulence, friction, extrusion, collision, and pressure drop. In some embodiments, the one or more external forces is determined by fiber geometry. In some embodiments, the plurality of fibers are hollow fibers. In some embodiments, the plurality of hollow fibers are configured into a hollow fiber membrane module, wherein the plurality of hollow fibers are grouped together to form a hollow fiber bundle, and wherein the hollow fiber bundle is packed into a tube shell. In some embodiments, the hollow fiber membrane bundle is surrounded by a shell extending longitudinally along the length of the bundle, forming a lumen side and a shell side of the hollow fiber membrane module. In some embodiments, the lumen forms a tubular structure. In some embodiments the space between the hollow fiber bundle and the tube shell is capped. In some embodiments, the hollow fiber membrane module is sterile. In some embodiments, the hollow fiber membrane module is single-use. In some embodiments, the portion of cells lysed is greater than about 90%.

### Methods for Isolating Products From Cells

The present disclosure provides methods of isolating intracellular products (e.g., polypeptides, viral particles or nucleic acid molecules) from a suspension of cells. In some embodiments, the nucleic acid molecules are closed-end nucleic acid molecules. In some embodiments, the closed-end nucleic acid molecules are closed-end double stranded DNA molecules. In some embodiments, the intracellular product is a polypeptide. In some embodiments, the polypeptide is an antibody or fragment thereof. In some embodiments, the polypeptide is an antigen binding protein or fragment thereof.

In one aspect, the disclosure provides a method for isolating an intracellular product of interest from a suspension of cells, comprising:
(i) flowing the suspension of cells comprising viral particles or nucleic acid molecules through a cartridge comprising a plurality of fibers or tubes, wherein the fibers or tubes have an inner diameter of no greater than 0.7 mm, and wherein the cells experience a pressure differential of less than 250 psi, thereby lysing a portion of the cells in the suspension to produce a mixed suspension comprising lysed cells and unlysed cells;
(ii) recirculating the suspension of partially lysed cells formed in step (i) through the cartridge for five or more additional cycles, wherein additional portions of the cells are lysed at each successive cycle to produce a suspension comprising lysed cells and cellular debris; and
(iii) separating the intracellular product of interest from the suspension comprising lysed cells and cellular debris, thereby isolating the intracellular product of interest.

In some embodiments, the suspension of cells is initially housed in a container that is connected to a cartridge. Examples of fibers and fibrous porous materials that can be used in the cartridge are discussed below. As set forth above, the cells in the suspension may experience a pressure differential as they are flowed through the cartridge, causing lysis of at least some cells and creating a suspension of partially lysed cells. The suspension of partially lysed cells can then be recirculated a number of cycles through the cartridge to cause lysis to more cells. The final suspension comprises a mixture of lysed cells and cellular debris.

The pressure differential (e.g., a pressure drop) experienced by cells flowing through the cartridge is the difference in total pressure between two points of a fluid carrying network. A pressure drop occurs when frictional forces, caused by the resistance to flow, act on a fluid as it flows through the tube. A piping network containing a high relative roughness (e.g., a membrane comprising pores) will affect the pressure drop. High flow velocities and/or high fluid viscosities result in a larger pressure drop across the cartridge. Low velocity will result in lower or no pressure drop. In some embodiments, the pressure differential is determined by the fiber geometry of the cartridge. In some embodiments, the pressure differential is defined by the difference in pressure at the cartridge inlet and pressure at the cartridge outlet. In some embodiments, the suspension of cells flowing through the cartridge experience a pressure differential of less than 1,000 psi. In some embodiments, the suspension of cells flowing through the cartridge experience a pressure differential of less than 500 psi. In some embodiments, the suspension of cells flowing through the cartridge experience a pressure differential of less than 250 psi. In some embodiments, the pressure differential is less than 200 psi. In some embodiments, the pressure differential is less than 150 psi. In some embodiments, the pressure differential is less than 100 psi. In some embodiments, the pressure differential is less than 50 psi. In some embodiments, the pressure differential is from about 5 psi to about 40 psi. In some embodiments, the pressure differential is from about 10 psi to about 40 psi. In some embodiments, the pressure differential is from about 15 psi to about 35 psi. In some embodiments, the pressure differential is from about 20 psi to about 30 psi.

The suspension of cells may be recirculated through the cartridge. The recirculation step re-introduces the suspension of partially lysed cells to the cartridge. In some embodiments, a cycle comprises circulating the suspension from the container through the cartridge, as depicted in step (ii). In some embodiments, a cycle comprises re-circulating the suspension of partially lysed cells to the cartridge. In some embodiments, the method comprises recirculating the suspension of partially lysed cells for at least one cycle. In some embodiments, the method comprises recirculating the suspension of partially lysed cells at least five, at least six, at least seven, at least eight, at least nine, at least ten or more additional cycles. In some embodiments, the suspension of partially lysed cells are recirculated through the cartridge for at least 10 cycles. In some embodiments, the suspension of partially lysed cells are recirculated through the cartridge for at least 20 cycles. In some embodiments, the suspension of partially lysed cells are recirculated through the cartridge for at least 30 cycles. In some embodiments, the suspension of partially lysed cells are recirculated through the cartridge for at least 40 cycles. In some embodiments, the suspension of partially lysed cells are recirculated through the cartridge for at least 50 cycles. In some embodiments, the suspension of partially lysed cells are recirculated through the cartridge from about 1 cycle to about 50 cycles. In some embodiments, the suspension of partially lysed cells are recirculated through the cartridge from about 10 cycles to about 50 cycles. In some embodiments, the suspension of partially lysed cells are recirculated through the cartridge from about 15 cycles to about 40 cycles. In some embodiments, the suspension of partially lysed cells are recirculated through the cartridge from about 20 cycles to about 30 cycles. In some embodiments, suspension of cells is flowed through a container in each successive cycle. In some embodiments, the container is a bioreactor. In some embodiments, the bioreactor is a closed system.

In some embodiments, each additional cycle produces additional portions of the cells are lysed at each successive cycle to produce a suspension comprising lysed cells and cellular debris.

In some implements, products that may be produced in a suspension of cells (e.g., host cells such as producer cells) include macromolecules such as polypeptides, polypeptide complexes, or polynucleotides. Non-limiting examples of polypeptides include antibodies, enzymes, and signaling peptides. In some embodiments, the product is a nucleic acid molecule. Particular nucleic acid include, but are not limited to DNA molecules RNA molecules (e.g., mRNA), and siRNA molecules.

In some embodiments, the product to be isolated is viral particle. In some such embodiments, the viral particle is an adenovirus (AV), adeno-associated virus (AAV) or a lentiviral particle. In some embodiments, the viral particles are produced by a triple transfection method. In some embodiments, the product to be isolated is a closed-end DNA for gene therapy.

The cells may comprise nucleic acid compositions as well as expression vectors containing the nucleic acids that are used to express the product. In some embodiments, the cells are induced to express the product. In some embodiments, the cells constitutively express the product. The nucleic acids can be put into expression vectors that contain the appropriate transcriptional and translational control sequences, including, but not limited to, signal and secretion sequences, regulatory sequences, promoters, origins of replication, selection genes, etc.

In some embodiments, the viral particle to be isolated may be a recombinant viral particle that includes a gene that expresses a protein of interest. In one embodiment, the viral particle is a rAAV that comprises a transgene expressing a product of interest. rAAV is a non-enveloped, single-stranded DNA virus which has been modified to be a replication-deficient entity, capable only of infecting cells and delivering DNA into their nuclei. A rAAV particle can comprise viral proteins and viral nucleic acids of the same serotype or a missed serotype. More than 40 serotypes of AAV are currently known, and new serotypes and variants of existing serotypes continue to be identified. See Gao et al., PNAS, 2002, 99(18): 11854-6; Gao et al., PNAS, 2003, 100(10):6081-6; and Bossis et al., J. Virol., 2003, 77(12):6799-810. Different AAV serotypes are used to optimize transduction of particular target cells or to target specific cell types within a particular target tissue. In some embodiments, the viral particles comprise an AAV serotype. In some embodiments, the AAV serotype comprises at least one of: AAV1, AAV2, AAV3a, AAV3b, AAV4, AAV5, AAV6, AAV7, AAV8, AAV9, AAV10, AAV11, AAV12, AAV13 and variants thereof, or chimeras, or hybrids thereof. In some embodiments, the viral particles are rAAV particles that are derived from an AAV serotype, including without limitation, AAV1, AAV2,AAV3a, AAV3b, AAV4, AAV5, AAV6, AAV7, AAV8, AAV9, AAV10, AAV11, AAV12, AAV13 and variants thereof, or chimeras, or hybrids thereof. In some embodiments, the rAAV viral particles are produced by a triple transfection method. In some embodiments, the viral particles are rAAV particles produced by triple transfection method, wherein the triple transfection method comprises transfecting a host cells with one or more plasmids comprising: (i) one or more nucleic acids encoding AAV packaging genes (such as nucleic acid encoding an AAV Rep protein and a nucleic acid encoding an AAV Cap protein); (ii) a nucleic acid encoding a gene of interest; (iii) a nucleic acid encoding at least one Adhelper virus gene. In some embodiments, the viral particles are rAAV particles produced by a transient transfection method. In some embodiments, the viral particles are rAAV particles produced by transfection of the host cell with one or more plasmids comprising: (i) one of more nucleic acids encoding AAV packaging genes (such as nucleic acid encoding an AAV Rep protein and a nucleic acid encoding an AAV Caps protein) and (ii) a nucleic acid encoding a gene of interest In some embodiments, the viral particles are rAAV particles produced by transfection of the host cell with one or more plasmids comprising: (i) one of more nucleic acids encoding AAV packaging genes (such as nucleic acid encoding an AAV Rep protein and a nucleic acid encoding an AAV Caps protein); (ii) a nucleic acid encoding a gene of interest; (iii) one or more nucleic acids encoding AAV ITRs (inverted terminal repeats) (e.g., two AAV ITRs), and optionally (iv) a nucleic acid encoding at least one Adhelper virus gene. In some embodiments, the viral particles are rAAV particles expressed from a producer cell line (PCL). In some embodiments, the PCL comprises a host cell stably integrated with: (i) one of more nucleic acids encoding AAV packaging genes (such as nucleic acid encoding an AAV Rep protein and a nucleic acid encoding an AAV Caps protein); (ii) a nucleic acid encoding a gene of interest: (iii) one or more nucleic acids encoding AAV ITRs (inverted terminal repeats) (e.g., two AAV ITRs). In some embodiments, the viral particles are rAAV particles expressed from a PCL that is further infected with an Adhelper virus (e.g., an adenovirus) that expresses one of more helper genes. In some embodiments, the rAAV particles comprise full capsid. In some embodiments, the rAAV particles are full capsid. In some embodiments, mechanical cell lysis, as provided in this disclosure, improves the fraction of full capsid titer in total capsid titer, compared to chemical lysis (e.g., using a detergent). In some embodiments, the full capsid titer in an rAAV preparation produced using mechanical lysis (e.g., using a mechanical lysis device or method as described herein), is about 2-fold to about 20-fold higher, compared to that in an rAAV preparation produced using chemical lysis (e.g., using a detergent, such as PS-20). In some embodiments, following purification, the percent of full capsid in total capsid titer at least about 50%, at least about 60%, at least about 70%, at least about 80%, at least about 90%, at least about 95%, at least about 99%. In some embodiments, following purification, the percent of full capsid is about 50%-95%, about 50%-80%, about 60%-90%, about 50%-70%. In some embodiments, the percent of full capsid is from about 1% to about 60%, such as about 10%, about 15%, about 20%, about 25%, about 30%, about 40%, about 45%, about 50%, about 55%, about 59%. In some embodiments, following purification, the percent of full capsids in total capsid titer is from about 1% to about 60%, such as about 10%, about 15%, about 20%, about 25%, about 30%, about 40%, about 45%, about 50%, about 55%, about 59%. In some embodiments, following purification, the percent of full capsids in total capsid titer is from about 1% to about 10%, such as about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%.

Intracellular product (e.g., viral particles or nucleic acid molecules or polypeptides) may be isolated from the suspension comprising lysed cells and cellular debris through a number of methods, including column chromatography, cesium chloride (CsCl) gradients, and the like. For example, a plurality of column purification steps can be used, such as purification over an anion exchange column, an affinity column and/or a cation exchange column. See, for example, International Publication No. WO 02/12455. In some embodiments, the viral particles or nucleic acid molecules are separated from the suspension comprising lysed cells and cellular debris, thereby isolating the viral particles or nucleic acid molecules.

In some embodiments, the total time to complete steps (i) and (ii) is from about 1 hour to about 6 hours. In some embodiments, the total time to complete steps (i) and (ii) is from about 1 hour to about 4 hours. In some embodiments, the total time to complete steps (i) and (ii) is from about 2 hours to about 4 hours.

The suspension of cells may be grown in any container that permits the cultivation of a population of cells. In some embodiments, the container is operably linked to a cartridge to form a closed system. In some embodiments, the cartridge is comprised of a PVC shell or a polycarbonate. In some embodiments, the container is a bioreactor. In some embodiments, the bioreactor is operably linked to a cartridge. In some embodiments, the container (e.g., bioreactor) operably linked to the cartridge forms a closed system. In some embodiments, the suspension of cells may be grown in a first container, then transferred to a second container that is operably linked to a cartridge. In such embodiments, the second container is operably linked to the cartridge. In some embodiments, the closed system further comprises at least one tubing line that is operably linked to the container and the cartridge, wherein the cell suspension flows between the container and the cartridge through the tubing. In some embodiments, the tubing is rubber. In some embodiments, the tubing is silicone.

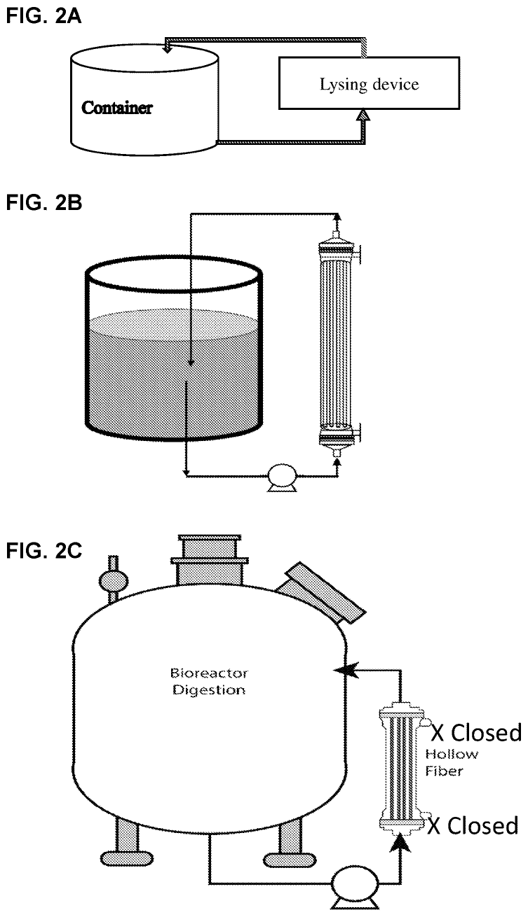

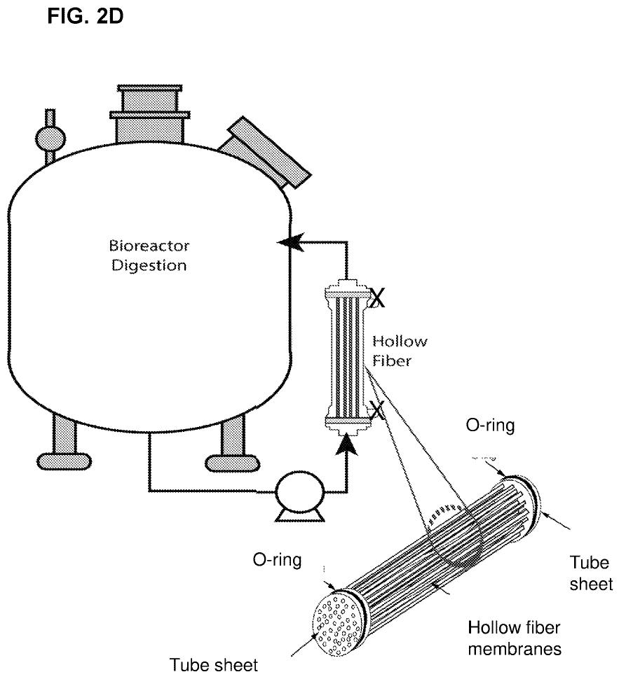

FIG. 2A is a schematic of a container that is operably linked to a cartridge such that the container and the cartridge forms a closed system. In one embodiment, the cartridge includes a hollow fiber membrane module (FIG. 2B). FIGS. 2C and 2D are schematics of a bioreactor operably linked to a hollow fiber membrane module. The Xs in FIGS. 2C and 2D indicate capped outlets.

In some embodiments, the cartridge comprises a plurality of hollow fibers, wherein the fibers comprise a lumen side and a shell side. In some embodiments, the plurality of hollow fibers are configured into a hollow fiber membrane module, wherein the plurality of hollow fibers are grouped together to form a hollow fiber bundle, and wherein the hollow fiber bundle is packed into a tube shell.

In some embodiments, the cells to be lysed are eukaryotic cells. In some embodiments, the cells are mammalian cells. In some embodiments, the mammalian cells are at least one of: CHO cells, HEK293 cells, Vero cells, HeLa cells, MDCK cells, BHK cells, or A549 cells. In some embodiments, the cells are derived from a human or a human cell line. In some embodiments, the human cell or human cell line is HEK293 or HeLa. In some embodiments, the cells are yeast cells. In some embodiments, the cells are insect cells.

In some embodiments, the cells are prokaryotic cells. In some embodiments, the prokaryotic cells are E. coli cells.

In some embodiments, the cells to be lysed are at least or about 15.5 μm in diameter. In some embodiments, the cells to be lysed are at least or about 16.9 μm in diameter. In some embodiments, the cells to be lysed are at least or about 20 μm in diameter. In some embodiments, the cells to be lysed are from about 10 μm to about 20 μm in diameter.

In some embodiments, the cells to be lysed are at high cell density. In some embodiments, the cells to be lysed are at high viable cell density. In some embodiments, the cells to be lysed are at a concentration of about 0.1 million cells/mL to about 200 million cells/mL. In some embodiments, the cells to be lysed are at a concentration of about 0.5 million cells/mL to about 100 million cells/mL. In some embodiments, the cells to be lysed are at a concentration of about 0.5 million cells/mL to about 50 million cells/mL. In some embodiments, the cells to be lysed are at a concentration of about 0.5 million cells/mL to about 50 million cells/mL. In some embodiments, the cells to be lysed are at a concentration of about 1 million cells/mL to about 50 million cells/mL. In some embodiments, the cells to be lysed are at a concentration of about 10 million cells/mL to about 50 million cells/mL. In some embodiments, the cells to be lysed are at a concentration of about 10 million cells/mL to about 25 million cells/mL. In some embodiments, the cells to be lysed are at a concentration of about 1 million cells/mL to about 25 million cells/mL. In some embodiments, the cells to be lysed are at a concentration of about 5 million cells/mL to about 15 million cells/mL. In some embodiments, the cells to be lysed are at a concentration of about 50 million cells/mL to about 100 million cells/mL. In some embodiments, the cells to be lysed are at a concentration of about 100 million cells/mL to about 200 million cells/mL. In some embodiments, the cells to be lysed are at a concentration of at least about 0.1 million cells/mL, at least about 0.5 million cells/mL, at least about 1 million cells/mL, at least 2 million cells/mL, at least 3 million cells/mL, at least 4 million cells/mL, at least about 5 million cells/mL, at least 6 million cells/mL, at least 7 million cells/mL, at least 8 million cells/mL, at least 9 million cells/mL, at least about 10 million cells/mL, at least about 12 million cells/mL, at least about 15 million cells/mL, at least about 20 million cells/mL, at least 25 million cells/mL, at least 30 million cells/mL, at least 40 million cells/mL, at least 45 million cells/mL, at least 46 million cells/mL, at least 47 million cells/mL, at least 48 million cells/mL, at least 49 million cells/mL, at least about 50 million cells/mL, at least 51 million cells/mL, at least 52 million cells/mL, at least 53 million cells/mL, at least 54 million cells/mL, at least 55 million cells/mL, at least about 75 million cells/mL, at least about 100 million cells/mL, at least 150 million cells/mL, at least about 200 million cells/mL. In some embodiments, the cells to be lysed are at a concentration of 2 million cells/mL. In some embodiments, the cells to be lysed are at a concentration of 4 million cells/mL.

Flow rate is the measure of the volume of liquid that moves in a certain amount of time. In some embodiments, the suspension of cells is flowed through the cartridge at a flow rate. The flow rate, in some embodiments, is based on various cartridge factors (e.g., fiber geometry or hollow-fiber geometry), such as the fiber diameter, fiber length, number of fibers. For instance, the flow rate can be determined based on the shear rate at the wall of the fibers. Pressure drop through the cartridge and the flow rate can be related to the shear rate. In some embodiments, the suspension is flowed through the cartridge at a shear rate of at least about 2000 s⁻¹. In some embodiments, the shear rate is from about 50 s⁻¹ to about 100,000 s⁻¹ or greater.

In some embodiments, the suspension of cells is flowed through the cartridge at a flow rate of from about 10 L per minute to about 100 L per minute. In some embodiments, the suspension of cells is flowed through the cartridge at a flow rate of at least about 0.1 mL per minute, of at least about 1 mL per minute, of at least about 10 mL per minute, of at least about 100 mL per minute, of at least about 1 L per minute, of at least about 10 L per minute, of at least about 100 L per minute. In some embodiments, the suspension of partially lysed cells is flowed through the cartridge at a flow rate of from about 10 L per minute to about 100 L per minute. In some embodiments, the suspension of partially lysed cells is flowed through the cartridge at a flow rate of from about 20 L per minute to about 50 L per minute. In some embodiments, the suspension of partially lysed cells is flowed through the cartridge at a flow rate of at least about 0.1 mL per minute, of at least about 1 mL per minute, of at least about 10 mL per minute, of at least about 100 mL per minute, of at least about 1 L per minute, of at least about 10 L per minute, of at least about 100 L per minute. In some embodiments, the suspension of cells is flowed only through the inside of the fibers or tubes.

The cells can be held in a container that is operably linked to the cartridge such that the container and the cartridge forms a closed system. In some embodiments, the container is temperature regulated. Temperature regulating a container allows the cells to be incubated or held at a set temperature. In some embodiments, the temperature is the temperature at which the cells are grown. In some embodiments, the temperature is from about 25° C. to about 37° C. In some embodiments, the temperature is about 25° C., about 26° C., about 27° C., about 28° C., about 29° C., about 30° C., about 31° C., about 32° C., about 33° C., about 34° C., about 35° C., about 36° C., or about 37° C. In some embodiments, the container is a bioreactor. In some embodiments, the bioreactor is set to a temperature. In some embodiments, the temperature of the bioreactor is at about 25° C. to about 37° C. In some embodiments, the temperature of the bioreactor is at about 25° C. In some embodiments, the temperature of the bioreactor is at 25° C. In some embodiments, the temperature of the bioreactor is at about 37° C. In some embodiments, the temperature of the bioreactor is at 37° C. In some embodiments, the bioreactor is not held at a temperature.

Separating viral products (e.g., rAAV) or nucleic acid molecules from the target suspension comprising lysed cells and cellular debris can be achieved by targeting various properties such as size or charge of the product or molecule to be separated. One example of a separating technique is affinity capture. In some embodiments, the target viral product or nucleic acid molecule comprises a moiety for affinity capture. The moiety for affinity capture is recognized by a matrix or resin that comprises a binding moiety (e.g., a protein, a polypeptide, or a ligand) that allows for the viral product or nucleic acid to be retained by the immobilized matrix or resin. In some embodiments, the separating comprises affinity capture. Another example of a separating technique is ion exchange. Ion exchange uses a matrix (e.g., resin or beads) that is charged (e.g., negatively charged or positively charged). in certain buffer conditions, the target viral product or nucleic acid molecule win interact preferentially with the charged matrix and is retained whereas the remainder of the suspension does not. For example, anion exchange uses a positively charged resin to bind negatively charged molecules. In some embodiments, the separating comprises ion exchange. In some embodiments, the separating comprises anion exchange.

### Fibers or Tubes

In one aspect, the disclosure provides a method for isolating an intracellular product of interest from a suspension of cells, comprising:
(i) flowing the suspension of cells comprising the intracellular product of interest (e.g., viral particle or nucleic acid) through a cartridge comprising a plurality of fibers or tubes, thereby lysing a portion of the cells in the suspension to produce a suspension of partially lysed cells;
(ii) recirculating the suspension of partially lysed cells formed in step (i) through the cartridge for five or more additional cycles, wherein additional portions of the cells are lysed at each successive cycle to produce a suspension comprising lysed cells and cellular debris; and
(iii) separating the intracellular product of interest from the suspension comprising lysed cells and cellular debris, thereby isolating the product of interest.

Fibers or tubes generally will have an inner diameter and an outer diameter. The space measured by the inner diameter may also be known the lumen. Therefore, the inner diameter may also be referred to as the lumen diameter. The flow path of the suspension of cells is through the inner section of the fibers or tubes. The inner diameter can have a profound impact on the flow velocity of the cellular suspension. In some embodiments, the fibers or tubes have an inner diameter of no greater than 0.7 mm. In some embodiments, the fibers or tubes have an inner diameter of no greater than 0.6 mm. In some embodiments, the fibers or tubes have an inner diameter of no greater than 0.5 mm. In some embodiments, the fibers or tubes have an inner diameter of no greater than 0.4 mm. In some embodiments, the fibers or tubes have an inner diameter of no greater than 0.3 mm. In some embodiments, the fibers or tubes have an inner diameter of no greater than 0.2 mm. In some embodiments, the fibers or tubes have an inner diameter of from about 0.1 mm to about 0.7 mm. In some embodiments, the fibers or tubes have an inner diameter of from about 0.2 mm to about 0.5 mm. In some embodiments, the fibers or tubes have an inner diameter of from about 0.2 mm to about 0.3 mm. In some embodiments, the fibers or tubes have an inner diameter of about 0.2 mm. In some embodiments, the fibers or tubes have an inner diameter of about 0.25 mm. In some embodiments, the fibers or tubes have an inner diameter of about 0.3 mm.

In some embodiments, the suspension of cells flowing through the cartridge experience a pressure differential of less than 1,000 psi. In other embodiments, the suspension of cells flowing through the fibers or tubes experience a pressure differential of less than 750 psi. In other embodiments, the suspension of cells flowing through the fibers or tubes experience a pressure differential of less than 500 psi. In other embodiments, the suspension of cells flowing through the fibers or tubes experience a pressure differential of less than 300 psi. In other embodiments, the suspension of cells flowing through the fibers or tubes experience a pressure differential of less than 250 psi. In other embodiments, the suspension of cells flowing through the fibers or tubes experience a pressure differential of less than 200 psi. In other embodiments, the suspension of cells flowing through the fibers or tubes experience a pressure differential of less than 150 psi. In other embodiments, the suspension of cells flowing through the fibers or tubes experience a pressure differential of less than 100 psi. In other embodiments, the suspension of cells flowing through the fibers or tubes experience a pressure differential of less than 75 psi. In other embodiments, the suspension of cells flowing through the fibers or tubes experience a pressure differential of less than 60 psi. In other embodiments, the suspension of cells flowing through the fibers or tubes experience a pressure differential of less than 50 psi. In other embodiments, the suspension of cells flowing through the fibers or tubes experience a pressure differential of less than 40 psi. In other embodiments, the suspension of cells flowing through the fibers or tubes experience a pressure differential of less than 30 psi. In other embodiments, the suspension of cells flowing through the fibers or tubes experience a pressure differential of less than 20 psi. In other embodiments, the suspension of cells flowing through the fibers or tubes experience a pressure differential of from about 5 psi to about 100 psi. In other embodiments, the suspension of cells flowing through the fibers or tubes experience a pressure differentia from about 5 psi to about 40 psi. In other embodiments, the suspension of cells flowing through the fibers or tubes experience a pressure differential of from about 10 psi to about 40 psi. In other embodiments, the suspension of cells flowing through the fibers or tubes experience a pressure differential of from about 15 psi to about 35 psi. In other embodiments, the suspension of cells flowing through the fibers or tubes experience a pressure differential of from about 20 psi to about 30 psi. In some embodiments, the suspension of cells flowing through the fibers or tubes experience a pressure differential of about 50 psi, about 45 psi, or about 35 psi.

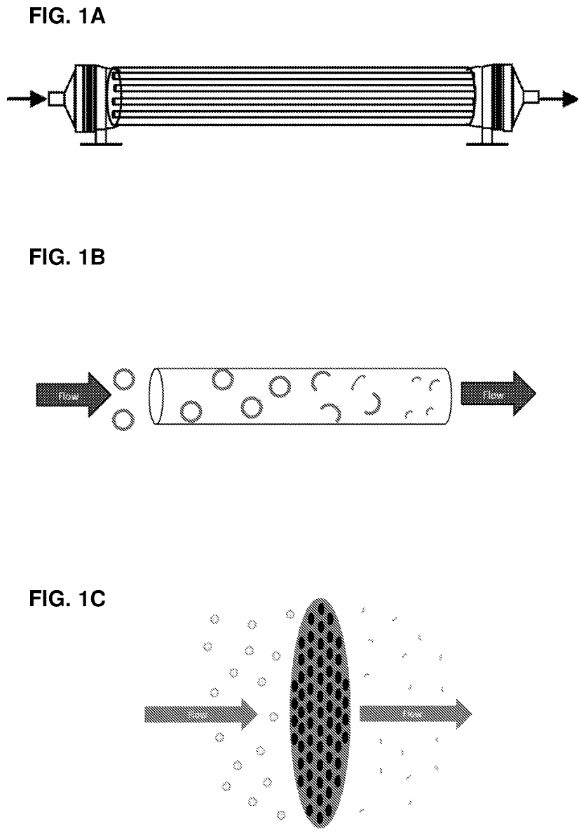

In some embodiments, the cartridge comprises a plurality of hollow fibers. In some such embodiments, the plurality of hollow fibers are grouped together to form a hollow fiber bundle, wherein the hollow fiber bundle is packed into a tube shell. In some embodiments, the cells flowing through the hollow fiber membranes experience a pressure differential as defined above. A hollow fiber membrane module is a liquid-tight housing in which a plurality of fibers are arranged in parallel (FIG. 1A).

Hollow-fiber modules comprise hollow-fibers that have various hollow fiber geometries. Hollow-fiber geometry describes fiber characteristics that influence mechanical cell lysis such as, but are not limited to, surface roughness, pore shape, pore diameter, and fiber diameter.

Hollow fiber modules comprise long, porous fibers (e.g., filaments) that have various diameters. The pores are embedded within the plurality of fibers. The fibers have an inner diameter, an outer diameter, two ends with a length between said ends, at least one end of each fiber open for fluid entrance or exit, and the fibers arranged parallel to one another. The hollow fiber module has two compartments within the cartridge. The intracapillary (IC) space describes the space within the hollow fibers, and the extracapillary (EC) space describes the space surrounding the hollow fibers. The hollow fibers generally have a membrane coated on the outside of a porous fiber support. The individual fibers are bundled together by cementing the fibers together. In some embodiments, the hollow fiber membrane is comprised of a modified polyethersulfone, polysulfone, polyethersulfone, mixed cellulose ester, and the like. In some embodiments, the hollow fiber membrane is comprised of a formulation of materials, such as polysulfone, polyether sulfone, polyvinylidene fluoride (PVDF), hydrophilized PVDF membrane. In some embodiments, the hollow fiber membrane is comprised of polypropylene, urethanes, polyethylene terephthalate, polyether ether ketone. In some embodiments, the hollow fiber membrane is comprised of ceramics, metallic membranes, or mixed matrix devices.

In some embodiments, the hollow fibers have an inner diameter of no greater than 0.7 mm. In some embodiments, the hollow fibers have an inner diameter of no greater than 0.6 mm. In some embodiments, the hollow fibers have an inner diameter of no greater than 0.5 mm. In some embodiments, the hollow fibers have an inner diameter of no greater than 0.4 mm. In some embodiments, the hollow fibers have an inner diameter of no greater than 0.3 mm. In some embodiments, the hollow fibers have an inner diameter of no greater than 0.2 mm. In some embodiments, the hollow fibers have an inner diameter of from about 0.2 mm to about 0.5 mm. In some embodiments, the hollow fibers have an inner diameter of from about 0.2 mm to about 0.3 mm. In some embodiments, the hollow fibers have an inner diameter of about 0.2 mm. In some embodiments, the hollow fibers have an inner diameter of about 0.25 mm. In some embodiments, the hollow fibers have an inner diameter of about 0.3 mm.

In some embodiments, the pores of the hollow fibers are embedded on a membrane. In some embodiments, the pores have a diameter of about 0.1 mm to about 1 mm. In some embodiments, the pores have a diameter of about 0.3 mm to about 1 mm. In some embodiments, the pores have a molecular weight cut-off of about 0.3 mm to 0.5 mm. In some embodiments, the pores have a diameter of about 100 kDa to about 700 kDa. In some embodiments, the pores have a molecular weight cut-off of about 100 kDa to about 500 kDa. In some embodiments, the pores have a molecular weight cut-off of about 100 kDa to about 300 kDa. In some embodiments, any of the preceding pore diameters are average pore diameters over the bundle of fibers. In some embodiments, the hollow fibers do not have pores.

In some embodiments, the shell side of the hollow fiber module is fully capped thus, directing the flow through the lumens (e.g., the intercapillary space) and not through the pores of these fibers. In some embodiments, the extracapillary space is capped. In some embodiments, the flow directs the suspension of cells through the length of the fiber and not through the pores. In some embodiments, the flow through tubular structures causes intentional damage to cells in the suspension. An example of an implementation is depicted in FIG. 1B. In some embodiments, the intentional damage is cell lysis.

Sterilization is the process of making something free from contaminants such as other cells. In some embodiments, the hollow fiber membrane module is sterile. In some embodiments, the hollow fiber membrane module is single-use. In some embodiments, the hollow fiber membrane module is not single-use. In some embodiments, the hollow fiber membrane module is reused. In some embodiments, the hollow fiber membrane module is cleaned prior to being reused.

In some embodiments, the hollow fibers are cellulose or synthetic polymers. In some embodiments, the cellulose-based hollow fibers are made from cellulose acetate or cuprammonium rayon. In some embodiments, the synthetic polymers are made from polysulfone, polyamide, or polyacrylonitrile. In some embodiments, the hollow fibers made from 2-methacryloyloxyethyl phosphorylcholine (MPC).

In some embodiments, the hollow fibers have a thickness of from about 0.1 mm to about 0.4 mm. In some embodiments, the hollow fibers have a thickness of from about 0.3 mm to about 0.4 mm.

In some embodiments, the length of the hollow fibers is from about 20 cm to about 150 cm. In some embodiments, the length of the hollow fibers is from about 50 cm to about 150 cm. In some embodiments, the length of the hollow fibers is from about 100 cm to about 150 cm.

A table of exemplary hollow fiber membrane modules that may be used in the methods described herein is included in Table 1.

TABLE 1
Commercially available hollow-fiber membrane modules

| Product | | | | | | |
|---|---|---|---|---|---|---|
| Information | Repligen ™ HF | | Cytiva ™ HF | | Planova HF ™ | |
| Length | 20 | cm | 30-60 | cm | 20 | cm |
| ID | 500 | μm | 500 | μm | 380 | μm |
| Pore size | 100-700 | kDa | 100 | kDa | 35 | nm |

### Fibrous Porous Medium or Material

In another aspect, the disclosure provides a method for isolating an intracellular product of interest from a suspension of cells, from cells, comprising:
(i) flowing a suspension of cells comprising the intracellular product of interest (e.g., viral particles or nucleic acid molecules) through a cartridge comprising a fibrous porous medium or material, thereby lysing a portion of the cells;
(ii) recirculating the suspension of partially lysed cells formed in step (i) through the cartridge for five or more additional cycles, wherein additional portions of the cells are lysed at each successive cycle;
(iii) separating the intracellular product of interest from the cells and cellular debris, thereby isolating the intracellular product of interest.

In some embodiments, the cartridge may comprise (a) a housing (b) a fibrous porous material. Non-limiting examples of porous material include nonwovens (spunbonds, melt-blown fibers, felts, wet-laid fibrous media), wovens (fabrics), membranes or any porous media. In some embodiments, the nonwoven material comprises a graded layer of non-woven fibers, cellulose, and diatomaceous earth. The non-woven fibers, in some instances, comprise polypropylene, polyethylene, polyester, nylon or mixtures thereof. In some embodiments, the nonwoven material comprises PET, glass fiber, polylactic acid, PVDF, PTFE, or PAN. In some embodiments, the pore size of the fibrous porous material comprises is about 10 μm to about 70 μm. In some embodiments, the pore size of the fibrous porous material is from about 20 μm to about 60 μm. In some embodiments, the pore size of the fibrous porous material is from about 30 μm to about 50 μm. In some embodiments, the pore size of the fibrous porous material is from about 15 μm to about 70 μm. In some embodiments, the pore size of the fibrous porous material is from about 20% to about 90% of the cell diameter. In some embodiments, the pore size of the fibrous porous material is from about 30% to about 90% of the cell diameter. In some embodiments, the pore size of the fibrous porous material is from about 50% to about 80% of the cell diameter. In some embodiments, the pore size of the fibrous porous material is about 70%, about 80%, about 90% of the cell diameter.

In some embodiments, the fibrous porous material is a nonwoven material. In some embodiments, the pore size of the nonwoven material is from about 10 μm to about 70 μm. In some embodiments, the pore size of the nonwoven material is from about 20 μm to about 60 μm. In some embodiments, the pore size of the nonwoven material is from about 30 μm to about 50 μm. In some embodiments, the pore size of the nonwoven material is from about 15 μm to about 70 μm. In some embodiments, the pore size of the nonwoven material is from about 20% to about 90% of the cell diameter. In some embodiments, the pore size of the nonwoven material is from about 30% to about 90% of the cell diameter. In some embodiments, the pore size of the nonwoven material is from about 50% to about 80% of the cell diameter. In some embodiments, the pore size of the nonwoven material is about 70%, about 80%, about 90% of the cell diameter. In some embodiments, the nonwoven material enables retention of at least about 60% (such as, at least about 70%, about 80%, about 90%) of lysed cells and cellular debris comprising a size at least about 0.1 μm, about 2 μm, about 3 μm, about 4 μm, about 5 μm, about 10 μm, about 11 μm, about 12 μm, about 13 μm, about 14 μm, about 15 μm, about 16 μm, about 17 μm, about 18 μm, about 19 μm, about 20 μm, about 30 μm, about 40 μm, about 50 μm, about 100 μm, about 200 μm, about 300 μm, or about 500 μm. In some embodiments, the flow directs the suspension of cells through the pores of the porous material. In some embodiments, the flow through the pores causes intentional damage to cells in the suspension. In some embodiments, the intentional damage is cell lysis. FIG. 1C depicts flow of a cellular suspension through a fibrous porous material.

### Analyzing Lysing Efficiency

Provided herein are techniques for analyzing lysing efficiency that are useful for the devices, apparatus and methods provided herein. When determining lysing efficiency, an initial sample is isolated, then compared to a sample collected post processing (e.g., post lysing). Lysis efficiency measures cell lysis and can be done by analyzing any number of parameters. For example, product release, viable cell count, viable cell density, percentage, percent lysis (e.g., percentage of lysis), relative titer, or intracellular protein release are parameters that can be measured by nucleic acid/protein based or microscopy-based assays to determine lysis efficiency. In some embodiments, the lysis efficiency is measured by assessing an initial viable cell density, a final viable cell density, and determining the percent reduction between initial and final viable cell densities (e.g., a 90% lysis efficiency in this method corresponds to a 90% reduction between the initial and final viable cell densities).

Percentage lysis efficiency can also be expressed as percentage lysed (e.g., percentage lysis or percentage cells lysed). For example, percentage lysed may be determined by assaying for the viable cell count and/or viable cell density before and after processing. The decrease in viable cell count or viable cell density indicates the degree of cell lysis. In some embodiments, the percentage lysed achieved by mechanical cell lysis is about 15%, about 20% after 5 or less cycles. In some embodiments, the percentage lysed is about 40%, about 45%, about 50%, about 55%, about 60%, about 65%, about 70% after 10 cycles. In some embodiments, the percentage lysed is about 40%, about 45%, about 50%, about 55%, about 60%, about 65%, about 70%, about 75% after 15cycles. In some embodiments, the percentage lysed is about 40%, about 45%, about 50%, about 55%, about 60%, about 65%, about 70%, about 75%, about 80% after 20 cycles.

Intracellular products such as polypeptides (e.g., recombinant protein particles), viral particles, or nucleic acids which cannot be secreted and are located in specific cell locations necessitates cell lysis for product release. In some embodiments, the cells comprise producer cells. In some embodiments, the lysing efficiency is determined by product release. In some embodiments, the product is viral particles. In some embodiments, the viral particles are produced by triple transfection. In some embodiments, viral particles are rAAV particles. In some embodiments, the viral particles are produced by a triple transfection method. In some embodiments, the viral particles are lentiviral particles. In some embodiments, the viral particles are produced by triple transfection of the producer cells.

Relative titer is a measure of change of the viral titer of a sample. Relative titer may also be used as an indicator to measure the amount of virus released from cells. Relative titer may be expressed as a percentage or fold-change and may be determined by methods known in the art. Thus, relative titer is useful, for example, to determine the amount of viral titer in the supernatant or to determine the amount of virus released from the cells before and after sample processing (e.g., lysis such as mechanical cell lysis or surfactant based lysis). For example, relative titer may be calculated by sampling the supernatant for viral titer after processing and comparing it to the viral titer in the supernatant prior to lysis. As disclosed herein, determining the viral titer for the purposes of evaluating relative titer may be determined by using methods known in the art. In some embodiments, viral titer is determined by qPCR. In some embodiments, viral titer is determined by ELISA. In some embodiments, viral titer is determined by western blot. In some embodiments, the lysis efficiency is measured by assessing relative titer. In some embodiments, the relative titer achieved by mechanical cell lysis as disclosed by the methods described herein, is about 2-fold to 10-fold relative to the un-lysed control. In some embodiments, the relative titer achieved by mechanical cell lysis as disclosed by the methods described herein, is about 3-fold to 8-fold relative to the un-lysed control. In some embodiments, the relative titer achieved by mechanical cell lysis as disclosed by the methods described herein, is about 4-fold to 8-fold relative to the un-lysed control. In some embodiments, the relative titer achieved by mechanical cell lysis as disclosed by the methods described herein, is about 2-fold, about 3-fold, about 4-fold, about 5-fold, about 6-fold, about 7-fold, about 8-fold, about 9-fold, to about 10-fold relative to the un-lysed control. In some embodiments, the relative titer achieved is about 2-fold or greater relative to the un-lysed control after 10 cycles of lysis. In some embodiments, the relative titer achieved is about 2.5-fold, about 3-fold, about 3.5-fold, about 4-fold or greater relative to the un-lysed control after 15 cycles of lysis. In some embodiments, the relative titer is about 2.5-fold, about 3-fold, about 3.5-fold, about 4-fold, about 5-fold, about 6-fold or greater relative to the un-lysed control after 20 cycles of lysis.

Viral vectors (e.g., rAAV particles) or nucleic acids may be measured using various assays known in the art, including without limitation, measuring particle production or nucleic acid production amount and/or rate, quantifying protein, capsid, or nucleic acid production (e.g., after purification using any of the methods described herein), measuring transduction efficiency, production of nucleic acid or proteins (such as rAAV capsid proteins) (e.g., as assayed by Western blot or PCR based methods such as qPCR). rAAV quality may be further determined by techniques such as dynamic light scattering (DLS), size exclusion chromatography (SEC), and analytical ultracentrifugation (AUC). In some embodiments, the lysing efficiency is determined by rAAV quality, wherein quality is assessed by dynamic light scattering, size exclusion chromatography, or analytical ultracentrifugation.

Viable cell count is a method used in cell culture to determine the number of living cells in a culture and distinguishes between live and dead cells and may be expressed as a function of live cells versus total cells (e.g., total cells comprise both live and dead cells) or a function of initial live cell density compared to a final viable cell density. Non-limiting examples of how to determine viable cell count or viable cell count percentage include the use of trypan blue, a hemocytometer, or an automatic cell counter. In some embodiments, the lysing efficiency is determined by viable cell count percentage. In some embodiments, the lysis efficiency is measured by calculating a viable cell count percentage. In some embodiments, the viable cell count percentage is determined by assessing an initial viable cell density, a final viable cell density, and determining the percent reduction between initial and final viable cell densities (e.g., a 90% lysing efficiency as determined by this method corresponds to a 90% reduction between the initial and final viable cell count; e.g. a 90% lysing efficiency as determined by this method corresponds to 10% viable cell count percentage or 90% cells lysed). In some embodiments, the viable cell count percentage decreases after one or more cycles. In some embodiments, the viable cell count percentage decreases after one cycle. In some embodiments, the viable cell count percentage decreases at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30% after one cycle. In some embodiments, the viable cell count percentage further decreases after one or more additional cycles. In some embodiments, the viable cell count percentage decreases after one cycle, and further decreases after additional cycles. In some embodiments, the viable cell count percentage decreases at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30% with each additional cycle. In some embodiments, the viable cell count percentage does not decrease after the viable cell count percentage is determined to be about 1%, about 2%, about 3%, about 4%, about 5%, about 6%, about 7%, about 8%, about 9%, about 10%.

In some embodiments, the viable cell count percentage is from about 20% to about 99% after 5 cycles. In some embodiments, the viable cell count percentage is from about 5% to about 90% after 10 cycles. In some embodiments, the viable cell count percentage is from about 1% to about 70% after 20 cycles.

In some embodiments, the viable cell count percentage is about 50%, about 55%, about 56%, about 57%, about 58%, about 59% after one cycle. In some embodiments, the viable cell count percentage is about 20%, about 25%, about 30%, about 35%, about 40%, about 45%, about 50%, about 55%, about 56%, about 57%, about 58%, about 59% after two cycles. In some embodiments, the viable cell count percent is about 10%, about 11%, about 12%, about 13%, about 14%, about 15%, about 16%, about 17%, about 18%, about 19%, about 20% after four cycles. In some embodiments, the viable cell count percent is about 10%, about 11%, about 12%, about 13%, about 14%, about 15%, and less than 20% after five cycles.

In some embodiments, lysing efficiency is determined by intracellular protein release. In some embodiments, the protein is lactate dehydrogenase (LDH). Lactate dehydrogenase release may be measured by any number of methods. For example, the identity of protein is confirmed via antibody and the concentration released into the suspension versus contained intracellularly is determined by western blot.

In some embodiments, the methods described herein achieve at least greater than 70% lysing efficiency wherein lysing efficiency is measured by lactate dehydrogenase release, viable cell count percentage, or product release percentage per number of cycles. In some embodiments, the methods described herein achieve greater than 70% lysing efficiency wherein lysing efficiency is measured by lactate dehydrogenase release, viable cell count percentage, or product release percentage per number of cycles. In some embodiments, at least 70% lysing efficiency is achieved within 1 hours to 4 hours.

In some embodiments, the methods described herein achieve at least greater than 80% lysing efficiency wherein lysing efficiency is measured by lactate dehydrogenase release, viable cell count percentage, or product release percentage per number of cycles. In some embodiments, the methods described herein achieve greater than 80% lysing efficiency wherein lysing efficiency is measured by lactate dehydrogenase release, viable cell count percentage, or product release percentage per number of cycles. In some embodiments, at least 80% lysing efficiency is achieved within 1 hours to 4 hours. In some embodiments, at least 80% lysing efficiency is achieved within 18 cycles. In some embodiments, at least 80% lysing efficiency is achieved within 11 cycles. In some embodiments, at least 80% lysing efficiency is achieved within 8 cycles. In some embodiments, at least 80% lysing efficiency is achieved within 5 cycles.

In some embodiments, the methods described herein achieve at least greater than 90% lysing efficiency wherein lysing efficiency is measured by lactate dehydrogenase release, viable cell count percentage, or product release percentage per number of cycles. In some embodiments, the methods described herein achieve greater than 90% lysing efficiency wherein lysing efficiency is measured by lactate dehydrogenase release, viable cell count percentage, or product release percentage per number of cycles. In some embodiments, at least 90% lysing efficiency is achieved within 1 hours to 4 hours.

### EXAMPLES

The following examples are included for illustrative purposes only and are not intended to limit the scope of the invention.

### Example 1: Cell Lysis by Flowing a Suspension of Cells Through One or More Cartridges

In this example, mammalian cells that comprised intracellular recombinant adeno-associated virus (rAAVs) were lysed using methods disclosed herein. The goal of the experiment was to achieve maximum recovery of the rAAVs following lysis of the cells.

FIG. 1A is a schematic showing commercially available hollow-fiber (HF) modules which are a bundle of hollow fibers (e.g., hollowed out fibers) placed in a shell. The hollowed out fibers comprise pores in the walls of the fibers. The interior surface of the hollow fiber is described as the lumen or “lumen side”, and the exterior of the fiber is described as the “shell side” because the exterior of the fiber faces the module shell. These hollow fiber modules are available in various configurations (e.g., hollow fiber geometries). For example, variables include lumen diameter (e.g., inner diameter of the fiber or lumen ID), hollow fiber length, hollow fiber pore size, and total fiber area. These modules come in fully sterile and may be used in a single-use setup. These commercially available hollow-fiber modules are essentially a plug and play device with extremely stable and steady operation. Unlike the typical way of running these modules, the shell side of the module is fully capped thus, directing the flow only through the lumens and not through the pores of these fibers. FIG. 1B shows a diagram of how the cells are lysed through the hollow fibers, which lyses cells by pressure fluctuations, turbulence, and other external forces as described above. These fibers, or tubes come in a variety of cross diameters depending on the application and operation time. FIG. 2B shows a container (e.g., a bioreactor) operably linked to a hollow fiber module. The cells held within the container (e.g., a bioreactor) are recirculated through the hollow fiber module to achieve cell lysis.

Various commercially available hollow-fiber modules were used in the study (Table 1). These modules were chosen based on their different hollow-fiber geometries (e.g., lumen ID, length, pore size and other factors). Cells did not traverse through the pores since the shell side of the hollow fiber module was capped.

The assemblies consisted of using 24 size tubing or larger for flow rates higher than 100 mL/min to reduce the tubing effect on shearing, and experiments were performed using a peristaltic pump. Notably, the pump alone in the absence of hollow fibers had no significant or measurable contribution to the cell shearing. The pressures were monitored using Pendotech pressure sensor with the PMAT system. The hollow-fiber modules were cleaned with 0.1 N NaOH if they were to be reused, however, in most experiments the modules were only used once.

Lysing efficiency was quantified using various methods and techniques. ViCell was used to quantify the total cell count along with the viable cell count. The CEDEX Bio Analyzer was used to quantify the lactate dehydrogenase (LDH) levels, a cytosolic protein that is released upon necrosis. rAAV release was also quantified using qPCR for most of the experiments performed. rAAV quality data were obtained by processing bioreactors through the typical downstream process. The qualitative techniques include dynamic light scattering (DLS), size exclusion chromatography (SEC), and analytical ultracentrifugation (AUC).

### Results

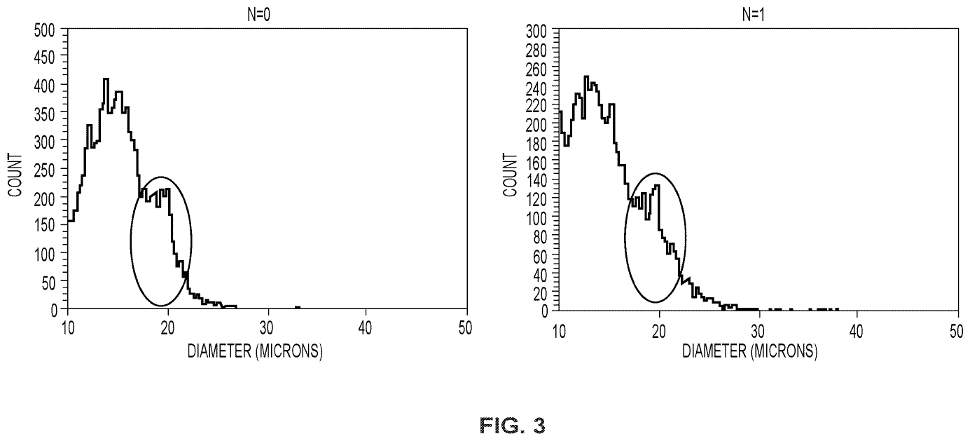

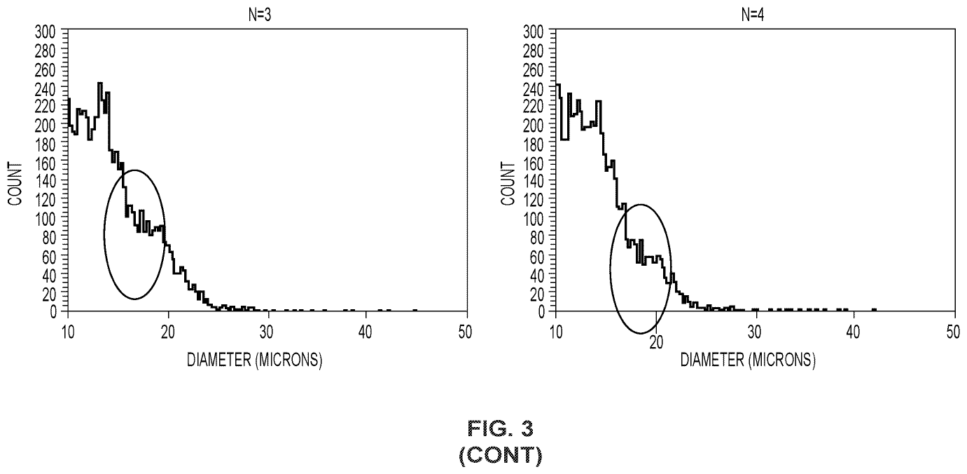

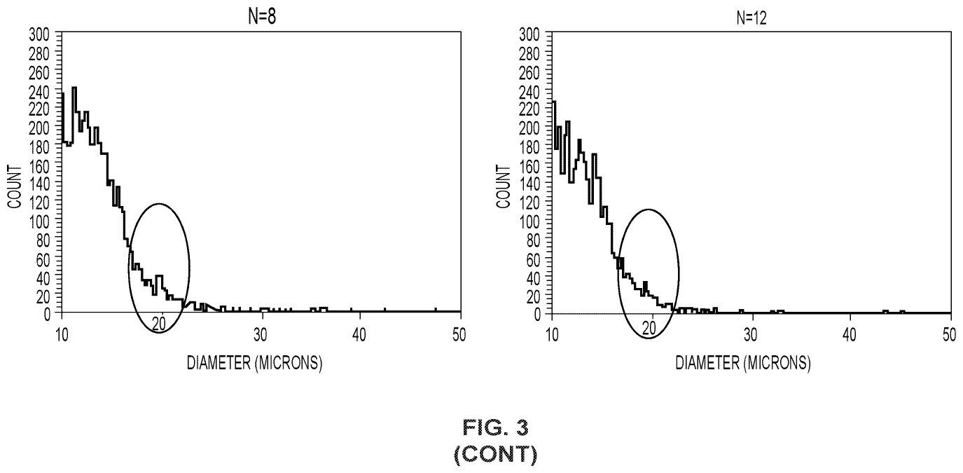

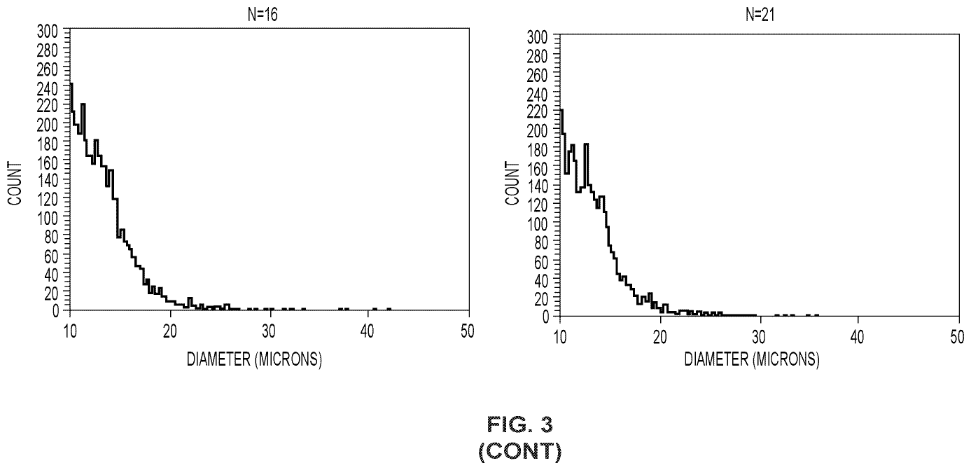

Un-lysed mammalian cells (HEK293) were observed to have a mean average diameter in the 20 μm range. Progression of cell lyses was monitored by the disappearance of the peak around 20 μm. The cell count cell distribution at various cycle numbers for HEK293 using Repligen™ hollow fiber module (HF) was observed to decrease as the cycle number increased until a count of 10 μm range after 16 cycles (FIG. 3). This lysis process was observed to be gradual because it required several cycles and ensured the rAAV was not damaged and sheared as well. The cells were also observed to be efficiently lysed without creating large quantities of cell aggregates.

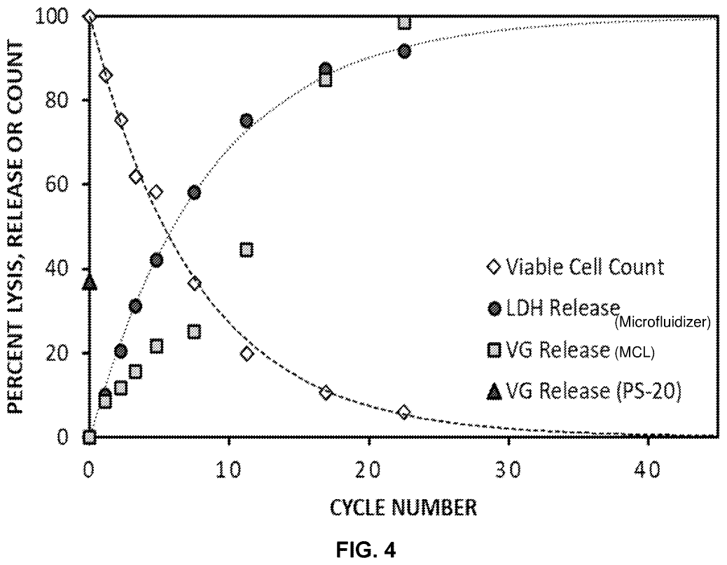

The percent lysed, for HEK293, according to LDH, viable cell count percentage, and product release percentage as a function of cycle number was determined for each parameter (FIG. 4). LDH release (using microfluidization) and the viable cell count percentage followed the same trend and was observed to be inversely proportional. The percentage cells lysed followed a traditional exponential decay model

$$( \frac{C}{{C}_{0}} = {e}^{- {k}_{C} N} ) ,$$

where C is the cell concentration, N is the number of cycles and Kc is a constant. A lysing efficiency of 95% was reached after 22 cycles. The product (rAAV) is released as a function of cycle number and reached the same value as the microfluidizer at a cycle number of 22. The hollow fiber mechanical cell lysis process released about 2.5× more rAAVs than using 0.2% PS-20.

Viable cell count percentage was also determined for a different mammalian cell line (HeLa). For HeLa cells, an 80% reduction in viable cell count was reached after 30 cycles (FIG. 5). Table 2 summarizes the results seen from HEK293 and HeLa cells. The initial viability and total cell density (TCD) were also reported in order to look further into the initial conditions' effect on lysis efficiency. For several very different TCD and initial cell viability, the lysing efficiency was within a 10% range after a cycle number of between 17-21. This further shows the independency of the mechanical lysis performance on the initial conditions.

TABLE 2
Table summarizing various small-scale
studies for 2 different cell types

| | Initial Cell | Initial | | | Lysis |
|---|---|---|---|---|---|
| | Diameter | TCD (cell | Initial Cell | Cycle | Efficiency |
| System | (μm) | 10⁶/mL) | Viability (%) | Number | (%) |
| HEK293 | 15.5 | 9.1 | 78 | 17 | 90 |
| HEK293 | 15.5 | 9.7 | 57 | 21 | 83 |
| HeLa cells | 18.0 | 0.7 | 86 | 18 | 76 |
| HeLa cells | 16.9 | 0.7 | 25 | 20 | 72 |
| Range: | 15.5-18.0 | 0.7-9.7 | 25-86 | 17-21 | 72-90 |

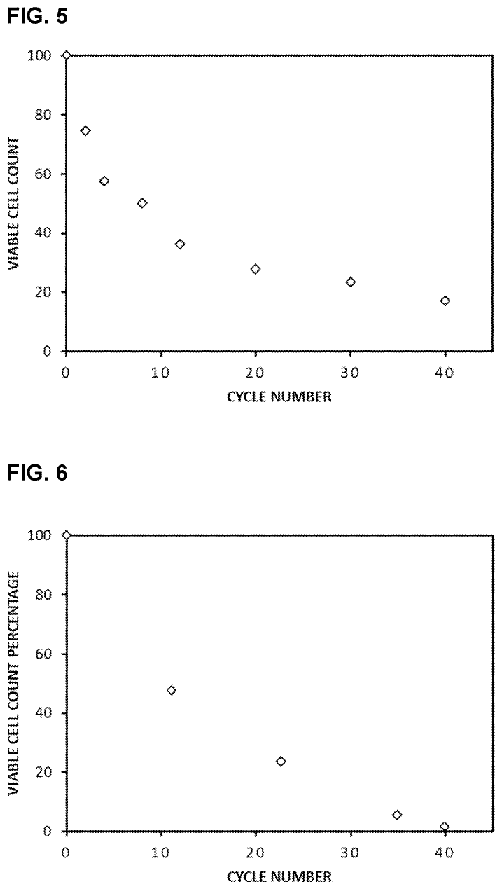

The same studies as described above were also done on Hela cells using a nonwoven material (FIG. 1C) with a pore size of 40 μm. Using a nonwoven material for cell lysis yields a decrease in viable cell count decreases from 100% to approximately 40% by cycle 10, and approximately 20% by cycle 20. (FIG. 6) As with the hollow fiber membrane approach, utilizing a cycle number-based optimization allows targeting of the desired lysing efficiency.

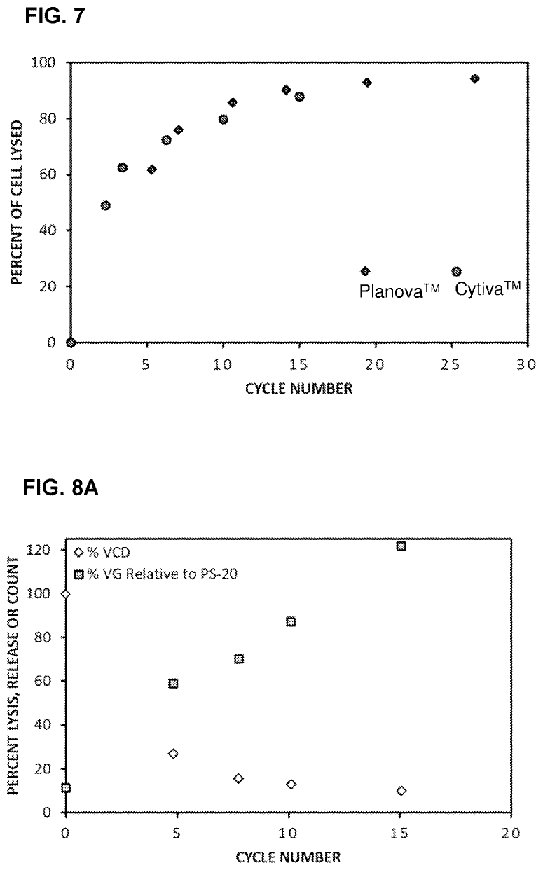

Next, various hollow-fiber modules were also tested using HEK293. In addition to the Repligen™ HF, Planova™ 35N HF and Cytiva™ HF were also used. The Planova™ gave a slightly better performance which may be attributed to the smaller fiber internal diameter (380 μm compared to 500 μm) (FIG. 7). The pore size was not observed to significantly impact cell lysis except for changing the friction factor of the lumens (Table 1, FIG. 7).

### Example 2: Mechanical Cell Lysis Executed at an Industrial Scale

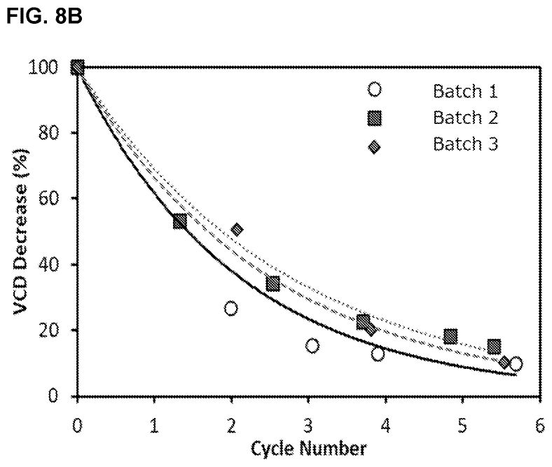

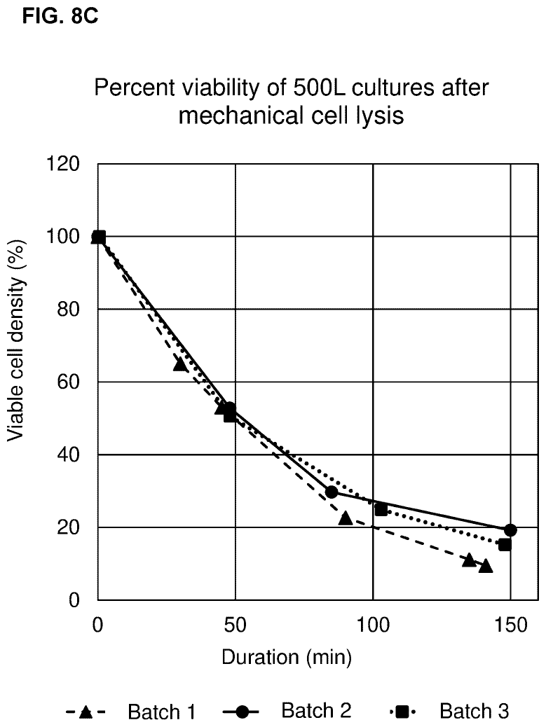

A closed system using hollow fiber membrane modules was assembled to demonstrate the mechanical cell lysis process at industrial scale (FIGS. 2A, 2C), using HEK293 cells expressing rAAV (Platform 1). For Platform 1, HEK293 cells were transiently transfected with AAV Rep and Cap genes, and a therapeutic gene of interest (GOI). The 500 L bioreactor was operably linked with two Cytiva™ Size 5 HF membranes (Cytiva™ UFP-100-C-5S) with the shell-side capped for both permeate outlets. The run was conducted at a flow rate of 40 liters per minute (LPM) with a differential pressure of 20 psi. The process took a total time of 2 hours and 10 minutes to achieve a lysing efficiency of 90%. The viable cell count (as assessed by percent cell lysis and percent viable cell density) and the vector genome release exhibited a similar and reproducible trend to the small-scale data (FIG. 8A, FIG. 8B). Additional large scale (500 L) cell cultures were processed by mechanical cell less to demonstrate that similar lysing efficiency was observed across multiple platforms (FIG. 8C).

Parameters as described in Table 3 were optimized and lysis efficiency was analyzed.

TABLE 3
Train conditions for cell lysis in closed
system using hollow fiber membrane module

| | | | | | Recorded |
|---|---|---|---|---|---|
| | | | | | average |
| | Module | | | | steady state |
| | Manufac- | Fiber | Total | Operating | pressure |
| System | turer | ID | Length | flow rate | (ΔP) |
| Train 1 | Cytiva ™ | 500 μm | 30 cm | 264 mL/min | 21 psi |
| Train 2 | Cytiva ™ | 500 μm | 30 cm | 100 mL/min | 16 psi |
| Train 3 | Cytiva ™ | 500 μm | 30 cm | 57 mL/min | 9 psi |
| Train 4 | Cytiva ™ | 500 μm | 120 cm | 87 mL/min | 20 psi |
| Train 5 | Planova ™ | 380 μm | 20 cm | 250 mL/min | 30 psi |
| Train 6 | Planova ™ | 380 μm | 20 cm | 100 mL/min | 7 psi |

### Results

By cycle 10, the rAAV titer released was about 80% of the titer released by PS-20; by cycle 15, the rAAV titer released exceeded the rAAV titer released by PS-20 (FIG. 8A).

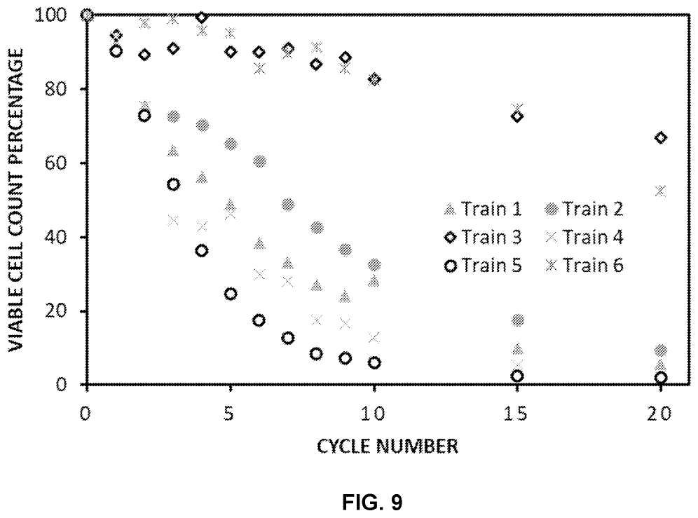

Train 5 performed the best, followed by Train 4 and then Train 1 (FIG. 9). This performance behavior suggested that pressure drop (i.e., the steady state pressure, psid, or the AP) was the leading mechanism in the cell lysis. The shear rate, which was proportional to the flow rate for Train 5 and Train 1 were observed to be the highest. Train 4 showed shear levels between Train 2 and Train 3 but outperformed both trains. Notably, it was also observed that if the material, fiber ID, total length were fixed, that the flow rate could be used to tune or vary the pressure drop (AP) (FIG. 9, Table 3).

### Example 3: Decreased Turbidity of Sample is Achieved With Mechanical Cell Lysis Compared to Surfactant Based Lysis

Methods: Exemplary rAAV was lysed and purified from 5 L cultures of HEK293 (Platform 1) or HeLa (Platform 2) cells using either mechanical cell lysis or PS-20. For Platform 2, HeLa cells were stably integrated with AAV Rep and Cap genes, a therapeutic genes of interest, and further infected with an Adhelper virus. Mechanical cell lysis (two Cytiva™ 20 cm² hollow fiber modules placed in parallel) was carried out for the indicated number of cycles. For detergent based lysis, 0.2% PS-20 was used. TFF was operated using Repligen™ Flat Sheet cassettes at an area of 0.02 m² at a cross flow rate flux of 200 L/hr/m² and a transmembrane pressure of 6 psi. The diafiltration buffer for both was 50 mM Tris with 380 mM NaCl at a pH of 8. The turbidity measurements were taken using the Orion AQ4500 Turbidity Meter.

Viable cell density (VCD) was determined, then used to calculate percentage lysed. Relative titer was determined by sampling the lysate, pelleting the un-lysed cells and assaying the supernatant for vg by qPCR.

### Results

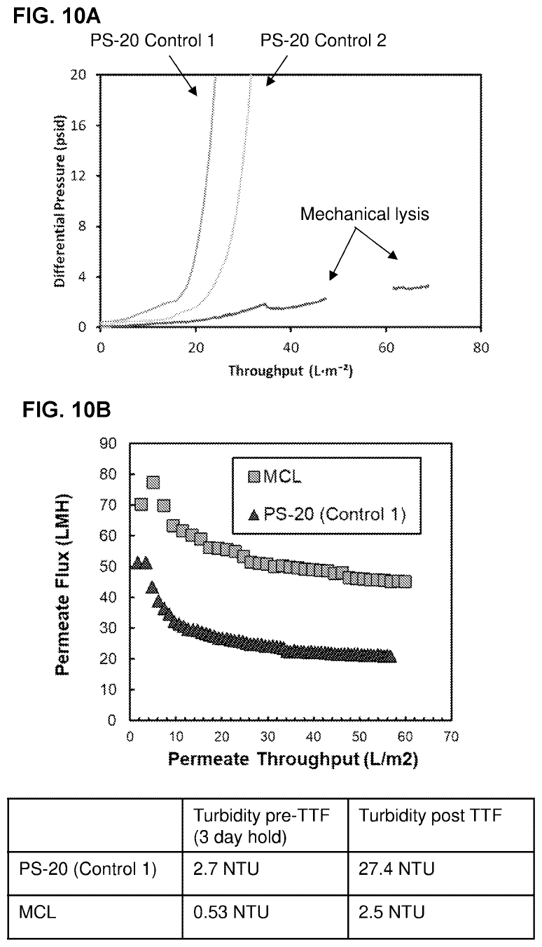

The exemplary rAAVs were extracted and purified from mechanically lysed Platform 1 cells, then analyzed by dynamic light scattering. Lysate obtained from mechanical cell lysis was observed to be less turbid and did not raise the differential pressure as significantly compared to lysate obtained from PS-20 lysed cells (FIG. 10A-10B), in depth filtration runs. PS-20 resulted in 60-70% (v/v) floc after 30 minutes settling, whereas mechanical lysis resulted in 20-30% (v/v) floc after 12 minutes settling (data not shown). Lysates were filtered using various commercially available depth filters, and the performance was compared (Table 4). Depth filtrations were observed to process more volume of mechanical lysis (MCL) processed lysates compared to PS-20 lysates (Table 4). MCL processed samples were also observed to have a denser floc sedimentation.

TABLE 4
Depth Filtration performance comparison

| | | | MCL + | PS-20 + |
|---|---|---|---|---|
| | MCL | PS-20 | Flocculation | Flocculation |
| Clarisolve ® 40 MS | N/A | N/A | 400 L/m² | 100 L/m² |
| D0HC | N/A | N/A | >70 L/m² | 25 L/m² |
| 3M ™ Harvest RC | 54 L/m² | 23 L/m² | N/A | N/A |

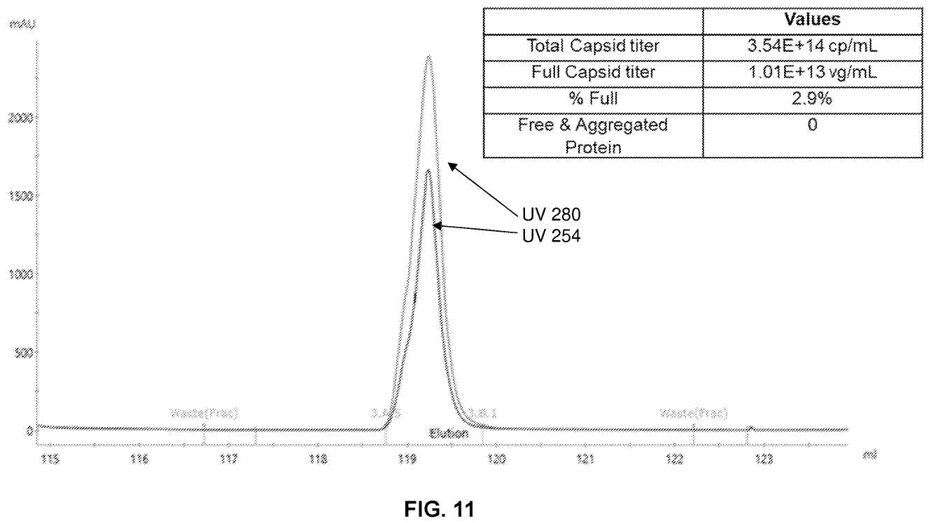

Capsid co-migrated with nucleic acid, indicating that a portion of the purified capsid was full capsid (FIG. 11). Similar analysis was performed with PS-20. Mechanical cell lysis extracted more (both total and full) of the exemplary rAAV capsid compared to PS-20 (Table 5). The observed percent full capsid titer (3% of total capsid isolated) obtained by mechanical cell lysis was also comparable to detergent-based methods (2% and 4% of total capsid isolated) (Table 5). Notably, the quantity of full capsid isolated from mechanical cell lysis (1.01 E+13 vg/mL) was greater than the quantity of full capsid (8.66 E+12 vg/mL and 5.77E+12 vg/mL) isolated from detergent based lysis, even in conditions where the percent full capsid obtained (4%) was higher than that obtained by mechanical cell lysis (3%).

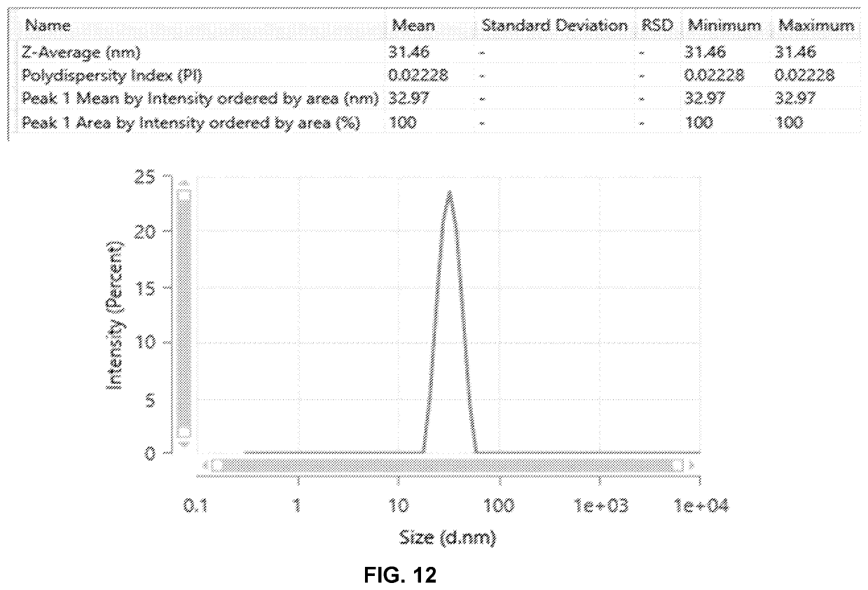

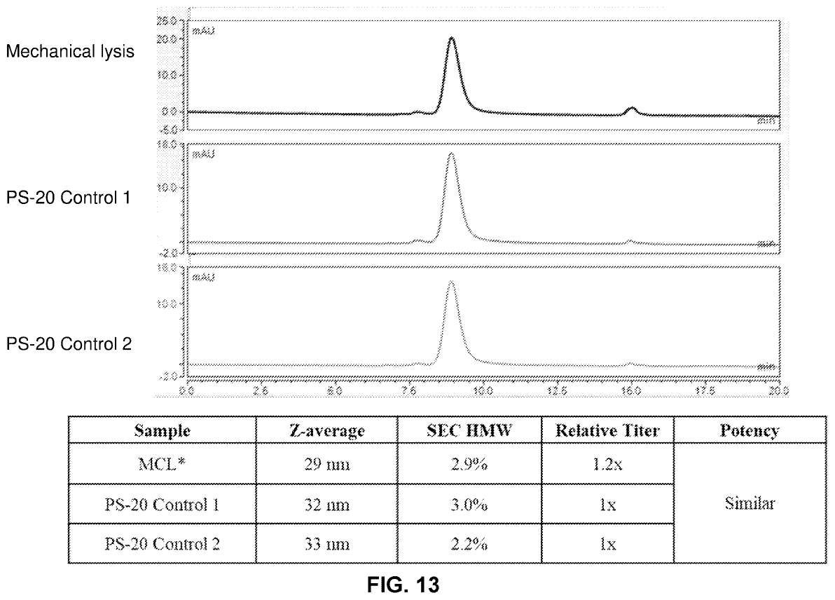

To validate that the exemplary rAAV obtained by mechanical cell lysis was at least similar in quality to the same rAAV obtained by detergent based methods, dynamic light scattering data was obtained for purified capsid. The purified Platform 1 capsid was determined to be approximately 30 nm in diameter and demonstrated low polydispersion, the rAAV particles within the sample did not have a broad size distribution (FIG. 12). Full capsid obtained by mechanical cell lysis displayed similar elution profiles compared to PS-20, indicating that the rAAV was consistent (e.g., at least of similar quality) to the rAAV obtained through detergent based lysis methods (FIG. 13).

TABLE 5
Capsid titer obtained by various cell lysis methods.

| | | PS-20 | PS-20 |
|---|---|---|---|
| | MCL | (Control 1) | (Control 2) |
| Total Capsid titer | 3.54E+14 | 2.38E+14 | 1.67E+14 |
| | cp/mL | cp/mL | cp/mL |
| Full Capsid titer | 1.01E+13 | 5.77E+12 | 8.66E+12 |
| | vg/mL | vg/mL | vg/mL |
| % Full | 3% | 2% | 4% |

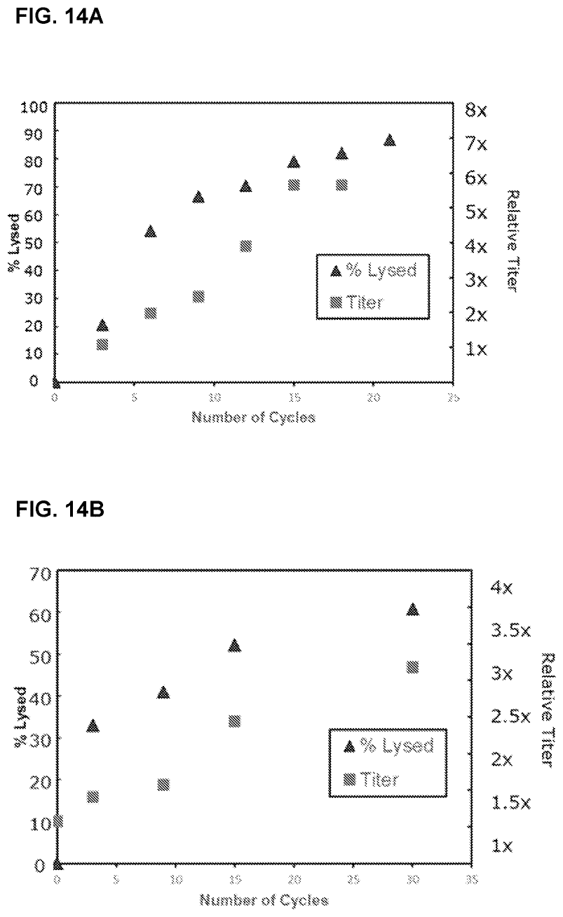

To demonstrate the reproducibility of mechanical cell lysis, further exemplary rAAV were released by mechanical cell lysis and purified from human cells (Platform 1 and Platform 2) (FIG. 14A, FIG. 14B). The downstream data assessing quality of the rAAV were observed to be similar between each platform compared to the control lysis (data not shown).

Relative release is one way to assess the efficiency of rAAV particle release from the cell. The relative release of rAAV particles from cells was measured by sampling the cell suspension before lysis, and again after lysis, via qPCR (FIGS. 13, 14A, and 14B).

Mechanical cell lysis across multiple platforms was observed to consistently achieve high rAAV particle release as indicated by the concomitant increase in relative titer and percent cell lysis. For example, it was observed that at least 50% cells were lysed by 10 to 15 cycles (FIGS. 14A and 14B). For Platform 1, nearly 80-90% cell lysis could be achieved by cycle 15 (FIG. 14A).

### Example 4: Mechanical Cell Lysis is Effective at Various Temperatures

Methods: Bioreactor temperature was regulated by water bath or warming blanket. Mechanical cell lysis was performed on 5 L cultures (Platform 1) using a hollow fiber membrane (Cytiva™ UFP-100-C-3MA), then capsid was purified by affinity chromatography followed by anion exchange. The percent full capsid was quantified by mass photometry.

Results: Temperature of mechanical cell lysis was investigated for its efficiency at various temperatures, and whether this impacted product yield and quality.

Without being bound by any specific theory, it was hypothesized that cell lysis conducted at lower temperatures increases product yield in three ways. The first is through cell efficiency. Cell membrane fragility increases at lower temperatures, translating to greater cell lysis and product release compared to lysing performed at higher temperatures. The second is through reducing the amount of product impurities. At higher temperatures, cells continue to produce rAAV that are more likely to be empty. The third is reducing the amount of product modifications. Higher temperature drives product post-translational modifications such as deamidation. Deamidation converts neutral residues to negatively charged residues, changing the anion exchange performance. Thus, product lysed at lower temperatures may be more productively recovered because it is less likely to be post-translationally modified.

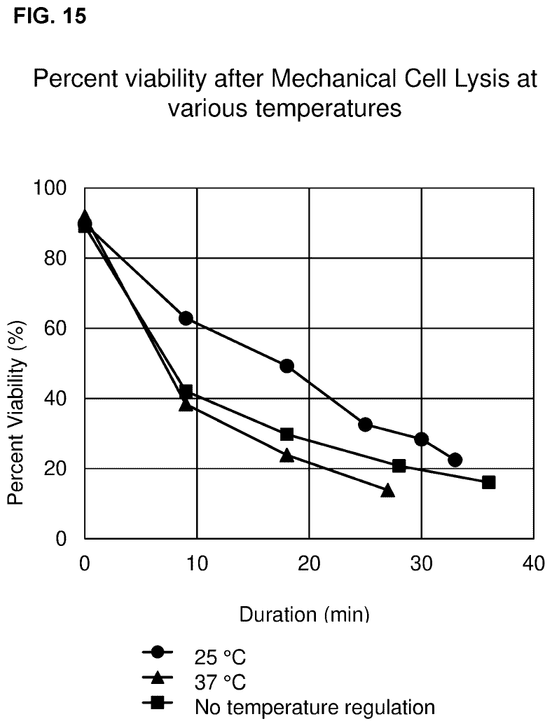

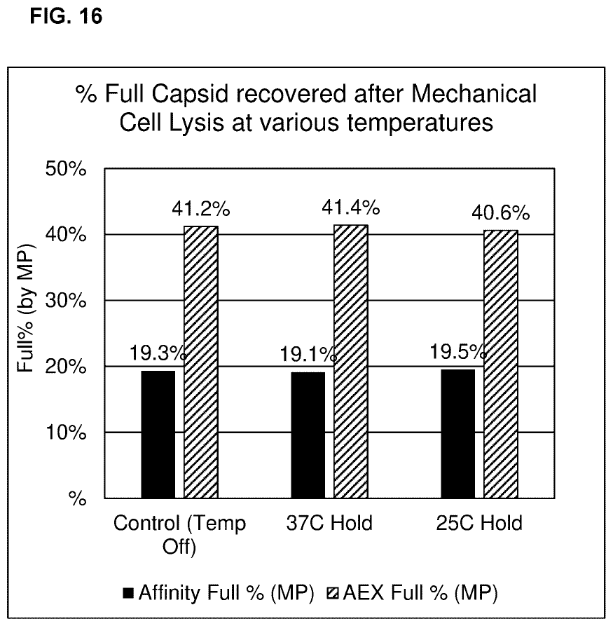

MCL was observed to achieve similar lysis efficiency between 37 and 25° degrees Celsius (FIG. 15). The percent of full product recovered after purification with affinity chromatography and anion exchange chromatography was also comparable (FIG. 16). The lowering of temperature for mechanical cell lysis did not adversely affect the cell lysis efficiency and product recovery.

### Example 5: Efficiency of Mechanical Cell Lysis at Various Starting Cell Densities

Methods: Platform 3 (producer line platform) are Hela cells 1.5 L of Platform 3 cells at 2 million cells/mL or 4 million cells/mL were processed by MCL using Repligen™ hollow-fiber PES filters (20 cm²) at pressures of 45-50 psi at 37° C.

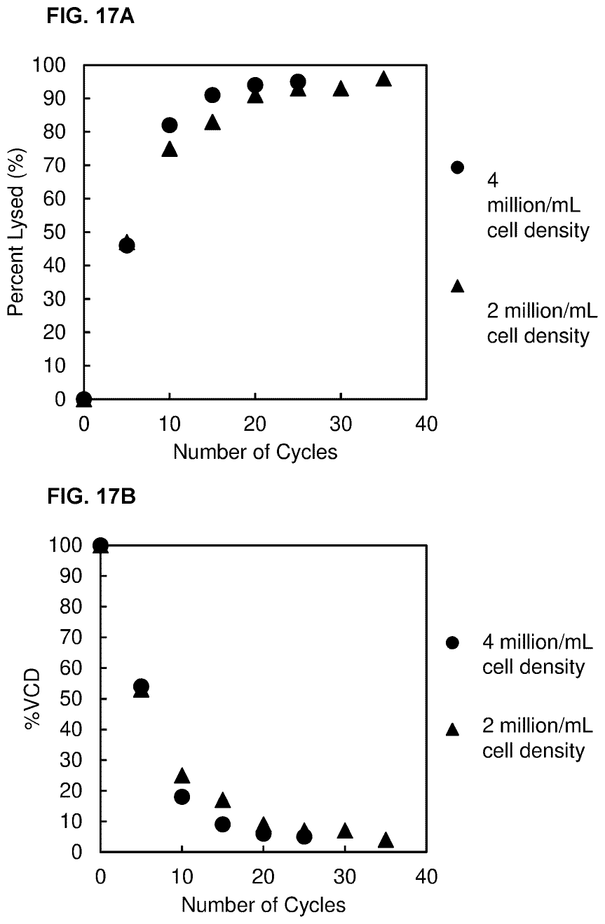

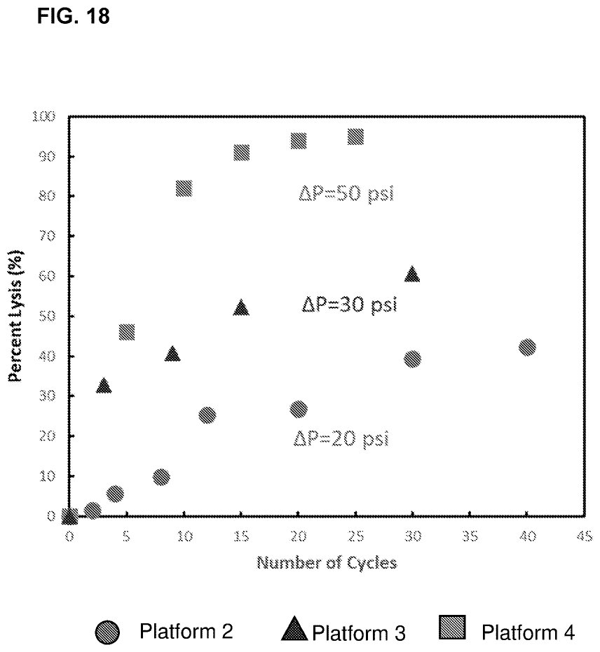

Results: The number of cycles required to reach comparable levels of cell lysis and viable cell density were similar for the 2 million cells/mL and the 4 million cells/mL culture (FIG. 17A, FIG. 17B). This result demonstrates that MCL was adapted to cultures of various densities.

### Example 6: Efficiency of Mechanical Cell Lysis is Related to the Pressure Drop

Methods: 5 L cell cultures for three different producer cell line platform cells (Platform 2, Platform 3, and Platform 4) were processed by MCL using Repligen™ hollow-fiber PES filters (20 cm²). Differing degrees of pressure drops were applied by modulating flow rate. The flow rates were 200 mL/min for 420 psi, 360 mL/min for 430 psi and 60 mL/min for 450 psi.

Results: Cells experiencing higher pressure drops were efficiently lysed compared to cells lysed using lower pressure drops (FIG. 18). Notably, lower pressure drops could not achieve equivalent levels of cell lysis despite extending the number of cycles (FIG. 18).

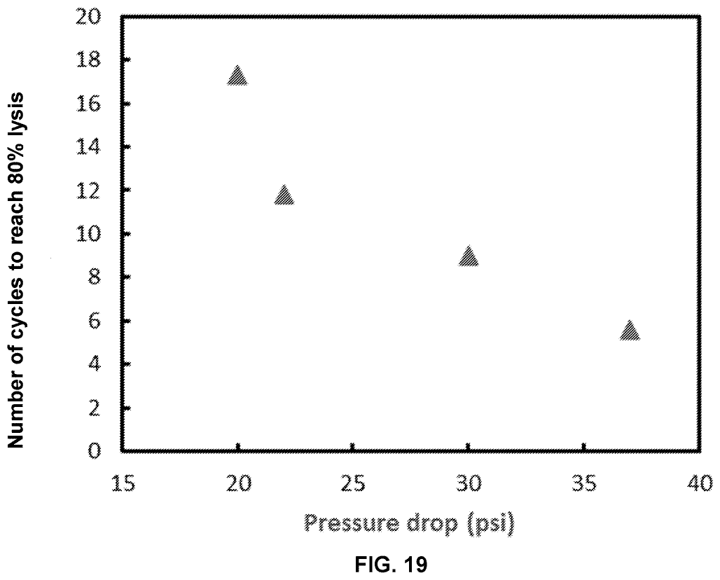

Methods: Cell culture at 5 L scale for HEK293 cells (Platform 1, a transient transfection platform) was also processed using various pressure drops and plotted against the number of cycles that it took to reach 80% lysis. Notably, it was observed that lysis using higher pressure drops required less cycles to achieve 80% lysis, whereas lower pressure drops required more cycles to achieve 80% lysis (FIG. 19). Pressure was modulated using flow rates as described above.

These results demonstrate that the pressure drop applied during mechanical cell lysis is an important determinant of the degree and the efficiency cell lysis.

The robustness of mechanical cell lysis is demonstrated for multiple volumes (5 L to 500 L), cell types, and AAV platforms. The mechanical lysis method was demonstrated to have various advantages as described above in both transient transfection (Platform 1, Platform 1A) and producer cell line (Platform 2, Platform 3, and Platform 4) based rAAV production systems. Platforms 1, 2, 3, and 4 each comprised a different gene of interest. The gene of interest for Platform 1A was same as that of Platform 3 but in case of Platform 1A—transient transfection based rAAV production system was tested, using HEK293 cells and in case of Platform 3 a producer cell line based rAAV production system was tested, using HeLa cells. The data presented indicates that this cell lysis method is both consistent and efficient for cell lysis and intracellular product release and recovery without the use of surfactant.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

What is claimed is:

1. A method for mechanically lysing cells, comprising flowing a suspension, the suspension comprising a plurality of cells, through a cartridge, the cartridge comprising a plurality of hollow fibers, wherein the plurality of cells is exposed to a pressure differential of about 20 to 50 psi, thereby lysing a portion of the plurality of cells in the suspension.

2. The method of claim 1, the method further comprising recirculating the plurality of cells through the cartridge for one or more cycles, thereby increasing a proportion of the plurality of cells that are lysed.

3. The method of claim 1, wherein the plurality of cells comprise producer cells that express viral particles.

4. The method of claim 1, the method further comprising introducing the plurality of cells to the cartridge by following a flow path.

5. The method of claim 1, wherein the plurality of cells are sheared inside the device.

6. The method of claim 1, the method further comprising applying one or more external forces to the plurality cells.

7. The method of claim 6, wherein the plurality of cells is exposed to one or more forces in the plurality of hollow fibers selected from the group consisting of turbulence, friction, extrusion, and collision.

8. The method of claim 1, wherein:
(a) the plurality of hollow fibers is configured into a hollow fiber membrane module;
(b) the plurality of hollow fibers is grouped together to form a hollow fiber bundle; and
(c) the hollow fiber bundle is packed into a tube shell.

9. The method of claim 8, wherein the hollow fiber membrane bundle is surrounded by a shell extending longitudinally along the length of the bundle, forming a lumen side and a shell side of the hollow fiber membrane module.

10. A method for isolating viral particles and nucleic acid molecules from a suspension of cells, the method comprising steps of:
(i) flowing the suspension of cells, the suspension of cells comprising particles, the particles comprising viral particles and nucleic acid molecules, through a cartridge, the cartridge comprising a plurality of hollow fibers, wherein the plurality of hollow fibers have an average inner diameter of about 0.3 mm to 0.7 mm, and wherein the cells are exposed to an average pressure differential of about 20 psi to 50 psi, thereby lysing a portion of the cells in the suspension to produce a mixed suspension comprising lysed cells and unlysed cells;
(ii) recirculating the mixed suspension formed in step (i) through the cartridge for one or more additional cycles, wherein additional proportions of the cells are lysed at each successive cycle to produce a target suspension, the target suspension comprising lysed cells and cellular debris; and
(iii) separating the particles from the target suspension, thereby isolating the particles.

11. The method of claim 10, wherein the viral particles comprise AAV particles.

12. The method of claim 10, wherein the suspension of cells comprises producer cells.

13. The method of claim 10, wherein the particles comprise lentiviral particles.

14. The method of claim 10, wherein the particles comprise a recombinant baculovirus expression vector.

15. A mechanical apparatus for lysing cells, comprising:
(a) a container capable of holding a suspension of cells;
(b) a cartridge comprising:
(i) a plurality of fibers; and
(ii) an extracapillary space outside the plurality of fibers; and
(c) a connector for connecting the container to the cartridge such that the container and cartridge form a closed system wherein, when operable, a suspension of cells flows one or more times through the container and the cartridge at a pressure differential of about 20 psi to 50 psi.

16. The apparatus of claim 15, wherein the plurality of fibers has a diameter up to about 0.7 mm.

17. The apparatus of claim 15, wherein the plurality of fibers has a diameter from about 0.1 mm to about 0.5 mm.

18. The apparatus of claim 15, wherein the plurality of fibers has a diameter from about 0.2 mm to about 0.3 mm.

19. The apparatus of claim 15, wherein the container is a bioreactor.

20. A cartridge for mechanically lysing AAV producing cells, comprising:
(a) a housing; and
(b) a plurality of pores comprising an inlet and an outlet operably positioned on the housing to permit the flow of an AAV producing cell suspension through the cartridge, wherein the configuration of the inlet and outlet is adapted to establish and maintain a pressure differential across the cartridge during operation, such that the AAV producing cell suspension flows through the cartridge experience a pressure drop sufficient to induce cell lysis, wherein the pores have a diameter up to about 1 mm.
